(19) United States
(12) Patent Application Publication
(10) Pub. No.: US 2010/0099122 A1
(43) Pub. Date: Apr. 22, 2010

(54) FERRIC REDUCTASE ACTIVITY OF HISTOPLASMA CAPSULATUM Y-GLUTAMYLTRANSFERASE

(76) Inventors: Jon P. Woods, Madison, WI (US); Robert Zarnowski, Madison, WI (US); Kendal G. Cooper, Salt Lake City, UT (US); Laura Schmitt-Brunold, Madison, WI (US)

Correspondence Address: WARF/BHGL, P.O. Box 10395, Chicago, IL, 60610, US

(21) Appl. No.: 12/543,269

(22) Filed: Aug. 18, 2009

Related U.S. Application Data
(60) Provisional application No. 61/091,616, filed on Aug. 25, 2008.

(51) Int. Cl. G01N 33/53; C07H 21/00; C12N 15/74; C12N 9/10; C12N 5/07; C07K 16/00; C12N 1/15

(52) U.S. Cl. 435/7.31; 536/23.2; 435/320.1; 435/193; 435/348; 530/387.1; 435/254.11; 435/7.1

(57) ABSTRACT

A novel gamma-glutamyltransferase gene and its encoded protein isolated from the fungus Histoplasma capsulatum are disclosed. The gene and protein are useful as novel fungicidal targets, or as research tools for conducting a new mode of glutamyl transfer and iron reduction. Compositions and methods that use the genes and proteins of this invention also disclosed, as are transgenic fungi that include the genes and proteins of the invention. Also disclosed are methods of using the novel genes and proteins.

### CROSS-REFERENCE TO RELATED APPLICATIONS

This invention claims priority to U.S. Provisional Patent Application Ser. No. 61/091,616, filed Aug. 25, 2008, which is herein incorporated by reference.

### GOVERNMENT INTERESTS

This invention was made with United States government support awarded by the National Institutes of Health, grants Nos. R01AI052303 and R01 HL055949. The United States government has certain rights in this invention.

### TECHNICAL FIELD

Isolated polypeptides with γ-glutamyltransferase activity, polynucleotides encoding the same, and vectors and recombinant microorganism expressing the same are provided. The polypeptides can be used in compositions and methods for the reduction of iron. The polypeptides can also be used to assay for antifungal compounds.

### BACKGROUND

Iron is an indispensable micronutrient for nearly all living organisms, where it acts as a cofactor of many enzymes involved in manifold vital cellular and physiological functions. In a mammalian host, iron is mainly maintained bound to highly specialized iron transport and iron storage proteins such as transferrin, lactoferrin and ferritin, and is sequestered in response to microbial infections. This essentially allows eliminating labile iron from plasma and extracellular tissues of the host, whereas the remaining pool of the metal can be ultimately bound to negatively charged molecules such as albumin. This system permits the mammalian host to maintain access to the metal in a soluble but non-reactive state while preventing the invading bacterial and fungal pathogens from acquiring iron and, consequently, from successful parasitism.

Iron has been implicated to have a critical role in infectious diseases (Weinberg, 1999, J. Eukaryot. Microbiol. 46: 231-238; Jung and Kronstad, 2008, Cell Microbiol. 10: 277-284. Excessive amounts of liberated iron may have certain hazardous implications. Iron overload severely compromises the iron withholding-based host defense strategy and contributes to the increase in susceptibility to some fungal and bacterial infections. Excessive amounts of iron impair the ability of phagocytes to kill invading pathogenic microorganisms. In addition, this element has the ability to initiate and catalyze the Fenton chemistry reactions, which lead to the formation of free oxygen radicals. These unstable molecules are highly reactive and can initiate cascades of lethal reactions in both the host and the infectious microorganism. As a consequence of all these properties, microbial pathogens have to compete for iron in the host so that they can multiply and establish a successful infection, but they also have to control and regulate iron acquisition tightly to prevent accumulation of excessive amounts of this metal (Kosman, 2003, Mol. Microbiol. 47: 1185-1197).

H. capsulatum is a human mycopathogen causing a deep systemic mycosis called histoplasmosis. Progressive disseminated histoplasmosis is a severe respiratory and systemic disease, mostly of the reticuloendothelial system, manifesting itself in the lungs, bone marrow, liver, and the spleen. The fungus enters and multiplies within human pulmonary macrophages in a unique phagosomal or phagolysosomal compartment, where it modulates, but does not completely reverse or block compartment acidification, and maintains a pH of approximately 6.5. Histoplasma infections generally produce a latent infection that periodically reactivates to produce active infections with varying clinical prognoses, with the greatest danger to immunocompromised patients. Iron is needed for both intracellular and in vitro growth of H. capsulatum. In response to infection with Histoplasma capsulatum, mammalian hosts specifically limit the levels of iron available to the fungus.

The fungus Histoplasma capsulatum (H. capsulatum) acquires iron via two general strategies. The first strategy relies on iron uptake before reduction and requires the presence of low molecular weight iron chelators called siderophores. H. capsulatum produces hydroxamate siderophores during mycelial and yeast phase growth in iron-depleted media. The fungus also uses xenosiderophores produced by other microbes. An alternative non-reductive route to acquire iron involves its gradual release from transferrin at acidic pH as well as cell wall-associated hemin binding. The second strategy relies on iron uptake after reduction and starts with extracellular iron reduction catalyzed by specific iron reductases or secreted external low weight molecular reductants (de Luca and Wood, 2000, Adv. Microb. Physiol. 43: 39-74). Reduction of ferric to ferrous iron followed by the transport of the latter form into the fungal cell provides an effective way to acquire iron from both inorganic and organic ferric salts, from Fe³⁺-loaded siderophores, or from host Fe³⁺-binding proteins.

H. capsulatum reduces iron in three ways: using glutathione-dependent ferric reductase (GSH-FeR); using low-molecular-weight non-enzymatic reductants; and using cell surface interactions (Timmerman and Woods, 1999, Infect. Immun. 67: 6403-6408). GSH-FeR is a proteinase K-susceptible, heat-labile secreted protein present in the high molecular weight fraction of supernatant. This enzyme reduces iron bound to siderophores and to iron binding proteins such as transferrin or hemin (Timmerman and Woods, 2001, Infect. Immun. 69: 7671-7678). GSH-FeR requires glutathione (GSH) for its activity and is reversibly inhibited by trivalent non-reducible iron analogues aluminum and gallium (Zarnowski and Woods, 2005, Microbiology-SGM 151: 2233-2240).

It is desirable to discover compositions that can be used in assays for the reduction of iron, and in assays for antifungal compounds. The present invention addresses these and related needs.

### BRIEF SUMMARY

Isolated polynucleotides are provided, which encode polypeptides comprising amino acid sequences that are at least 95% identical to the amino acid sequence of SEQ ID NO:2. These polypeptides have γ-glutamyltransferase activity. The isolated polynucleotides may be at least 95% identical to the nucleic acid sequence of SEQ ID NO:1. The isolated polynucleotides may encode the amino acid sequence of SEQ ID NO:2. The polynucleotides may encode polypeptides from Histoplasma capsulatum, and in particular they may encode polypeptides that have γ-glutamyltransferase activity in Histoplasma capsulatum. Polynucleotides that comprise the nucleotide sequences of the antisense strands of the above polynucleotides or parts thereof are provided as well.

Vectors are provided, which include the isolated polynucleotides of the present invention. The vectors may comprise recombinant expression cassettes that include promoter sequences operably linked to polynucleotides encoding polypeptides that comprise amino acid sequences that are at least 95% identical to SEQ ID NO:2, where the polypeptides have γ-glutamyltransferase activity. Also provided are host cells transformed with these vectors.

Isolated polypeptides are provided, which include amino acid sequences that are at least 95% identical to the amino acid sequence of SEQ ID NO:2, where the polypeptides have γ-glutamyltransferase activity. The isolated polypeptides may include the amino acid sequence of SEQ ID NO:2. These polypeptides may be extracellularly secreted after being expressed in host cells. The polypeptides may have γ-glutamyltransferase activity in Histoplasma capsulatum. The polypeptides may be involved in iron reduction. Also provided are antibodies immunologically specific for the polypeptides of the present invention. Methods for producing these polypeptides are provided, which include the steps of culturing host cells transformed with the vectors indicated above and recovering the expressed products. The expressed products may be recovered from the culture supernatants.

Transgenic organisms are provided, which include recombinant expression cassettes. The expression cassettes include promoter sequences operably linked to polynucleotides encoding polypeptides that include amino acid sequences that are at least 95% identical to the amino acid sequence of SEQ ID NO:2. The transgenic organisms may be transgenic Histoplasma capsulatum.

Methods are provided, which include: a) reacting polypeptides comprising amino acid sequences that are at least 95% identical to the amino acid sequence of SEQ ID NO:2 with glutathione to form cysteinylglycine; and b) reacting the cysteinylglycine with ferric iron (Fe³⁺) to form ferrous iron (Fe²⁺). The methods may further include the step of measuring the amount of formed ferrous iron, the amount of ferrous iron being proportional to γ-glutamyl transfer activity of the polypeptide. The methods may be performed extracellularly. In the practice of the methods, the polypeptide may be a γ-glutamyl transferase from Histoplasma capsulatum.

Provided are kits for use in analysis of iron reduction in a sample. The kits include: a) polypeptides comprising amino acid sequences that are at least 95% identical to the amino acid sequence of SEQ ID NO:2; and b) glutathione. The polypeptide and the glutathione may be present in one or more containers, and the polypeptide has a γ-glutamyltransferase activity. The kits may include polypeptides which are γ-glutamyl transferases from Histoplasma capsulatum.

Also provided are targets for fungicides, which include the gene products of nucleic acid sequences from a fungus coding for a protein having the activity of a γ-glutamyltransferase, the nucleic acid sequences comprising: a) a nucleic acid sequence having the nucleic acid sequence depicted in SEQ ID NO:1; or b) a nucleic acid sequence which can be derived by back-translation from the amino acid sequence depicted in SEQ ID NO:2, due to degeneracy of the genetic code; or c) functional equivalents of the nucleic acid sequences SEQ ID NO:1, which are at least 95% identical to SEQ ID NO:1.

Provided are methods for identifying substances with antifungal activity, where transcription, expression, translation, or activity of the gene products of the polynucleotides of the present invention is influenced. Those substances which reduce or block transcription, expression, translation or activity of the gene products are selected.

### BRIEF DESCRIPTION OF THE DRAWINGS

FIG. 1 is a graphical presentation of a gene encoding secreted γ-glutamyltransferase (GGT1) of H. capsulatum (A) and an image showing results of PCR amplification of GGT1 from genomic and cDNA templates (B).

FIG. 2 shows graphs (A, B, D) and images (B, C) of chromatographic analysis of Ggt1 purified from H. capsulatum G217B culture supernatants.

FIG. 3 shows graphs (A, B) of pH-dependent kinetics of the H. capsulatum Ggt1-catalyzed transfer and hydrolysis reactions of the artificial substrate γ-glutamyl-p-nitroanilide (GpNA).

FIG. 4 shows graphs (A, B) of Ggt1 enzymatic activity in high-molecular weight supernatant fractions of mutants with altered GGT1 expression levels.

FIG. 5 shows graphs (A, B) of the modulation of Ggt1 activity shows concordant alterations in extracellular iron reduction in H. capsulatum G217B culture supernatants.

FIG. 6 shows graphs (A, B) of ferric iron reducing activities of GSH () and CysGly (◯).

FIG. 7 is a graph illustrating how the process of Ggt1-catalyzed extracellular iron reduction is not dependent on pH due to complementary contribution of Ggt1 and cysteinylglycine activities.

FIG. 8 shows graphs (A, B) illustrating how the changes in GGT1 expression alter H. capsulatum growth and virulence.

FIG. 9 is a schematic diagram of a proposed model of Ggt1-assisted extracellular iron reduction in H. capsulatum, which involves two basic reactions.

### DETAILED DESCRIPTION OF THE PRESENTLY PREFERRED EMBODIMENTS

The present invention relates to the isolation and identification of a novel gene (“GGT1”) from Histoplasma capsulatum and the protein it encodes (“γ-glutamyltransferase”, “gamma-glutamyltransferase”, “GGT”, or “Ggt1”), and to a novel iron-reducing mechanism catalyzed by one of the products of the γ-glutamyltransferase reaction. GGT transfers a glutamyl residue from glutathione (a tripeptide consisting of γ-Glu-Cys-Gly) onto a substrate, thereby producing a dipeptide (Cys-Gly) that efficiently catalyzes the reduction of ferric iron (Fe³⁺). GGT enzymes have not been shown to be involved in iron reduction before. In particular, it has been discovered that the H. capsulatum-secreted γ-glutamyltransferase plays a role in extracellular iron reduction, as described in Zarnowski et al., 2008, Mol. Microbiol. 70: 352-368, which is herein incorporated by reference. The discovered γ-glutamyltransferase activity is complex and involves two reactions. First, γ-glutamyltransferase initiates enzymatic breakdown of GSH by cleavage of the γ-glutamyl bond and release of cysteinylglycine. Second, the thiol group of the released dipeptide reduces ferric to ferrous iron, resulting in efficient iron reduction over a broad pH range. The γ-glutamyltransferase genes and proteins of the present invention may thus be used for the control of γ-glutamyl transfer, for the control of iron reduction, as a source for iron reducing reagents and assays, and may be used as an antifungal drug target.

In some embodiments, the present invention provides a γ-glutamyltransferase gene (“GGT” or “GGT1”) and a γ-glutamyltransferase protein (“GGT protein” or “GGT polypeptide”) identified and cloned from the fungus Histoplasma capsulatum. In preferred embodiments, this invention provides extracellularly secreted γ-glutamyltransferase. For use in the present invention, the terms γ-glutamyltransferase and GGT also refer to polymorphic variants, mutants, alleles, and interspecies homologs of the γ-glutamyltransferase gene and protein cloned from Histoplasma capsulatum.

Generally, the nomenclature used herein and the laboratory procedures utilized in the present invention include molecular, biochemical, microbiological and recombinant DNA techniques that are well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Such techniques are thoroughly explained in the literature and are generally performed according to Sambrook et al., 1989, Molecular Cloning—A Laboratory Manual, Cold Spring Harbor Laboratory, Cold Spring Harbor, N.Y., N.Y.; Ausubel et al., 1993, Current Protocols in Molecular Biology, Volumes 1-3, John Wiley & Sons, Inc., Hoboken, N.J.; and Kriegler, 1990, Gene Transfer and Expression: A Laboratory Manual, Stockton Press, New York, N.Y.; Perbal, 1988, A Practical Guide to Molecular Cloning, 2^{nd} edition, John Wiley & Sons, New York, N.Y.; Watson et al., 1992, Recombinant DNA, 2^{nd} edition, Freeman & Co., New York, N.Y.; Bartlett and Stirling, 2003, PCR Protocols, 2^{nd} edition, Humana Press, Totowa, N.J.; all of which are incorporated herein by reference.

It has been discovered that the fungus Histoplasma capsulatum contains in its genetic material a region with a γ-glutamyltransferase gene. The nucleic acid sequence of the coding region of the γ-glutamyltransferase gene from Histoplasma capsulatum is shown as SEQ ID NO:1. The amino acid sequence of the γ-glutamyltransferase protein from Histoplasma capsulatum is shown as SEQ ID NO:2.

A “GGT polynucleotide” of the present invention: (1) comprises a nucleic acid sequence comprising a coding region of from about 50 to about 10,000 nucleotides, sometimes from about 100 to about 6,000 nucleotides, and preferably from about 500 to about 3,000 nucleotides, which hybridizes to SEQ ID NO:1 or the complement thereof under stringent conditions (as defined below), and conservatively modified variants thereof; (2) has substantial identity to the polynucleotide sequence of SEQ ID NO:1; and (3) encodes a GGT polypeptide.

As used herein, the phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid.

A “coding sequence” or “coding region” refers to a nucleic acid molecule having sequence information necessary to produce a gene product, when the sequence is expressed. The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be understood that the sequence includes the degenerate codons of the native sequence or sequences which may be introduced to provide codon preference in a specific host cell.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

In one embodiment of the present invention, provided are isolated nucleic acids that encode polypeptides which are involved in iron reduction, and most particularly in iron reduction affected by the fungus Histoplasma capsulatum. For example, the present invention provides isolated nucleic acids comprising polynucleotides at least 80% identical to a sequence as shown in SEQ ID NO:1. The present invention also provides isolated nucleic acids comprising polynucleotides at least 90% identical to a sequence as shown in SEQ ID NO:1. Yet in other examples, the present invention provides polynucleotides that are at least 95% identical to a sequence as shown in SEQ ID NO:1.

Isolated nucleic acids are provided, which include polynucleotide sequences that hybridize under stringent conditions to a sequence as shown in SEQ ID NO:1 or the complement thereof, where the nucleic acids encode polypeptides that encodes a γ-glutamyltransferase. In some embodiments, the nucleic acids of the present invention encode polypeptide sequences at least 80% identical to the polypeptide sequence as shown in SEQ ID NO:2. In other embodiments, the nucleic acids of the present invention encode polypeptide sequences that are at least 90% identical to the polypeptide sequence as shown in SEQ ID NO:2. In yet other embodiments, the nucleic acids of the present invention encode polypeptide sequences that are at least 95% identical to the polypeptide sequence as shown in SEQ ID NO:2. The polypeptides of the present invention have γ-glutamyl transfer activity as well as iron reduction activity in the presence of glutathione. The nucleic acids may be isolated from fungi, and in particular they may be isolated from Histoplasma capsulatum. Nucleic acids of the present invention can also be identified by their ability to hybridize under low stringency conditions (as described below) to nucleic acid probes having the sequence of SEQ ID NO:1 or the complement thereof, and fragments thereof. SEQ ID NO:1 is an example of the nucleic acids of the present invention.

Gamma-glutamyltransferase is an enzyme that catalyzes reversibly the transfer of a glutamyl group from a glutamyl-peptide and an amino acid to a peptide and a glutamyl-amino acid. In the IUBMB Enzyme Nomenclature GGT is EC 2.3.2.2

“Glutathione” (GSH) is a tripeptide consisting of γ-Glu-Cys-Gly

A “GGT polypeptide” of the present invention has substantial identity to the amino acid sequence of SEQ ID NO:2 and/or binds to antibodies raised against an immunogen comprising an amino acid sequence of SEQ ID NO:2. Preferred polypeptides of the present invention have γ-glutamyl transfer activity and are involved in iron reduction activity in the presence of glutathione. SEQ ID NO:2 is an example of the polypeptides of the present invention. Polypeptides of the present invention include polymorphic variants, mutants, and interspecies homologs of SEQ ID NO:2. Polypeptides of the present invention also include functional equivalents or fragments of SEQ ID NO:2. A preferred GGT polypeptide of the present invention has substantial identity to an amino acid sequence of SEQ ID NO:2 and/or is encoded by a polynucleotide that hybridizes under stringent conditions to SEQ ID NO:1 or the complement thereof. GGT polypeptides constitute a fairly large and ubiquitous group of enzymes involved in transpeptidation reactions, in which a γ-glutamyl residue is transferred from γ-glutamyl compounds to various acceptor molecules, such as amino acids, peptides or water (Kinlough et al., 2005, Methods Enzymol. 401: 426-449).

A functional fragment or functional equivalent or functional homolog of a polypeptide of the present invention is a polypeptide that is homologous to the specified polypeptide but has one or more amino acid differences from the specified polypeptide. A functional fragment or equivalent of a polypeptide retains at least some, if not all, of the activity of the specified polypeptide. In general, a GGT polypeptide functional homolog that preserves GGT polypeptide-like function includes any homolog in which residues at a particular position in the sequence have been substituted by other amino acids, and further includes the possibility of inserting an additional residue or residues between two residues of the parent protein as well as the possibility of deleting one or more residues from the parent sequence. Any amino acid substitution, insertion, or deletion is encompassed by the invention. In favorable circumstances, the amino acid substitution is a conservative substitution. Conservative substitutions whereby an amino acid of one class is replaced with another amino acid of the same type fall within the scope of the invention so long as the substitution does not materially alter the biological activity of the compound. For example, a functional equivalent of SEQ ID NO:2 shares the same amino acid sequence as SEQ ID NO:2 except for a few amino acid differences, e.g., substitutions, insertions, and/or deletions. When expressed in a fungus, e.g., a fungus from the Histoplasma genus, both SEQ ID NO:2 and its functional homolog have (i) γ-glutamyl transfer activity as well as (ii) iron reduction activity in the presence of glutathione. The functional equivalents of SEQ ID NO:1 are at least 61%, 62%, 63%, 64%, 65%, 66%, 67%, 68%, 69% and 70%, specifically at least 71%, 72%, 73%, 74%, 75%, 76%, 77%, 78%, 79%, 80%, preferably at least 81%, 82%, 83%, 84%, 85%, 86%, 88%, 88%, 89%, 90%, particularly preferably at least 91%, 92%, 93%, 94%, 95%, 96%, 97%, 98%, 99% identical to the nucleic acid sequence of γ-glutamyltransferase from H. capsulatum.

In one aspect, provided are methods for producing a polypeptide comprising an amino acid sequence that is at least 95% identical to the amino acid sequence of SEQ ID NO:2, comprising the steps of culturing a host cell transformed with a vector comprising the polynucleotide of SEQ ID NO:1, and recovering the expressed product. The method may further include recovering the expressed product from the culture supernatant.

Also provided are antibodies immunologically specific for all or part, e.g., an amino-terminal portion, of polypeptides of the present invention. “Antibodies” as used herein includes polyclonal and monoclonal antibodies, chimeric, and single chain antibodies, as well as Fab fragments, including the products of a Fab or other immunoglobulin expression library. With respect to antibodies, the term “immunologically specific” refers to antibodies that bind to one or more epitopes of a protein of interest, but which do not substantially recognize and bind other molecules in a sample containing a mixed population of antigenic biological molecules. The present invention provides antibodies immunologically specific for part or all of the polypeptides of the present invention, e.g., SEQ ID NO:2 or a fragment thereof. For example, the antibodies may be immunologically specific for polypeptides at least 80% identical to a sequence as shown in SEQ ID NO:2. The antibodies may be immunologically specific for all or part, e.g., an amino-terminal portion, of a GGT polypeptide encoded by an isolated nucleic acid that hybridizes under stringent conditions to a sequence as shown in SEQ ID NO:1 or the complement thereof. Accordingly, the present invention provides isolated antibodies or antibody compositions that specifically bind to a polypeptide having the amino acid sequence as shown in SEQ ID NO:2. In some embodiments, the antibodies may be monoclonal. Alternatively, the antibodies may be polyclonal. The antibodies of the present invention may be labeled using methods known in the art. A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or proteins for which antisera or monoclonal antibodies are available.

In one embodiment, provided are promoters, as segments of an isolated nucleic acid molecule for regulating expression of genes in transformed cells, and particularly in transformed fungal cells. In one example, the segments may comprise a portion of a gene that confers γ-glutamyl transfer activity. These segments typically commence at a location about 2500, preferably about 2000, bases upstream from a transcription initiation site of the gene that confers γ-glutamyl transfer activity and ends at a location about 250 bases downstream from the transcription initiation site. The segment is capable of increasing promoter activity of homologous or heterologous promoters in fungal species. In one example, the segment may include a 3′ untranslated region commencing at a stop codon for the gene's coding sequence, and ending at a location about 5000 bases downstream from the gene's transcription initiation site.

DNA segments for effecting expression of coding sequences operably linked to the segments are also provided. These DNA segments are typically isolated from a gene whose coding region hybridizes under stringent conditions with a coding region defined by SEQ ID NO:1. The DNA segment may comprise a promoter and a transcription initiation site, and it may include a polyadenylation signal. The DNA segment may be isolated from a Histoplasma capsulatum GGT gene.

In one embodiment of the present invention, provided are recombinant expression cassettes that include a promoter sequence operably linked to a nucleic acid of the present invention. In some embodiments, the nucleic acid comprises a polynucleotide sequence at least 80% identical to a polynucleotide sequence as shown in SEQ ID NO:1. The nucleic acid can be operably linked to the promoter in a sense or antisense orientation. In some examples, the promoter is a constitutive promoter, an inducible promoter, a tissue specific promoter, or a developmentally controlled promoter.

In another embodiment, provided are recombinant expression cassettes that include a promoter sequence operably linked to a nucleic acid comprising a polynucleotide sequence which hybridizes under stringent conditions to a sequence as shown in SEQ ID NO:1 or the complement thereof, where the nucleic acid encodes a GGT polypeptide. The term “recombinant” when used with reference, e.g., to a cell, or nucleic acid, protein, expression cassette, or vector, indicates that the cell, nucleic acid, protein, expression cassette, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

An “expression cassette” refers to a nucleic acid construct, which when introduced into a host cell, results in transcription and/or translation of a RNA or polypeptide, respectively. The expression cassette may include a nucleic acid comprising a promoter sequence, with or without a sequence containing mRNA polyadenylation signals, and one or more restriction enzyme sites located downstream from the promoter allowing insertion of heterologous gene sequences. The expression cassette is capable of directing the expression of a heterologous protein when the gene encoding the heterologous protein is operably linked to the promoter by insertion into one of the restriction sites. The recombinant expression cassette allows expression of the heterologous protein in a host cell when the expression cassette containing the heterologous protein is introduced into the host cell. Expression cassettes can be derived from a variety of sources depending on the host cell to be used for expression. For example, an expression cassette can contain components derived from a viral, bacterial, fungal, insect, plant, or mammalian source. In the case of both expression of transgenes and inhibition of endogenous genes (e.g., by antisense, or sense suppression) the inserted polynucleotide sequence need not be identical and can be “substantially identical” to a sequence of the gene from which it was derived.

Preferably the recombinant expression cassette allows expression in substantially all cells of an organism, such as a fungus. Examples of expression cassettes suitable for transformation of fungi can be found in PCT Patent Publication No. WO/1993/007277; European Patent No. EP0225078; Moralejo et al., 1999, Applied and Environmental Microbiology 65: 1168-1174, all of which are herein incorporated by reference.

The term “host cell” refers to a cell from any organism. Preferred host cells are derived from plants, bacteria, yeast, fungi, insects or various animals. The term “recombinant host cell” (or simply “host cell”) refers to a cell into which a recombinant expression vector has been introduced. It should be understood that the term “host cell” is intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Methods for introducing polynucleotide sequences into various types of host cells are well known in the art. The present invention provides host cells or progeny of host cells transformed with the recombinant expression cassettes of the present invention. The host cells may be fungal cells.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement of other transcription control elements (e.g. enhancers) in an expression cassette. Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “nucleic acid construct” or “DNA construct” is sometimes used to refer to a coding sequence or sequences operably linked to appropriate regulatory sequences and inserted into a expression cassette for transforming a cell. This term may be used interchangeably with the term “transforming DNA” or “transgene”. Such a nucleic acid construct may contain a coding sequence for a gene product of interest, along with a selectable marker gene and/or a reporter gene. The term “selectable marker gene” refers to a gene encoding a product that, when expressed, confers a selectable phenotype such as antibiotic resistance on a transformed cell. The term “reporter gene” refers to a gene that encodes a product which is easily detectable by standard methods, either directly or indirectly.

A “heterologous” region of a nucleic acid construct is an identifiable segment (or segments) of the nucleic acid molecule within a larger molecule that is not found in association with the larger molecule in nature. When the heterologous region encodes a fungal gene, the gene will usually be flanked by DNA that does not flank the fungal genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturallγ-occurring mutational events do not give rise to a heterologous region of DNA as defined herein. The term “DNA construct” is also used to refer to a heterologous region, particularly one constructed for use in transformation of a cell.

The term “vector” is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Antisense polynucleotides are also provided. For example, the invention provides antisense oligonucleotides complementary to SEQ ID NO:1 or a fragment thereof. In one embodiment, the antisense polynucleotides are less than about 200 bases in length.

In one embodiment of the present invention, provided are transgenic fungi having altered GGT expression and/or activity levels. The GGT expression and/or activity can be suitably upregulated (i.e. increased expression/activity) or downregulated (i.e. decreased expression/activity). In particular, the present invention provides transgenic fungi having enhanced γ-glutamyl transfer activity. Transgenic fungi of the present invention may include recombinant expression cassettes comprising a promoter operably linked to a nucleic acid of the present invention. The nucleic acid can be operably linked to a promoter sequence in a sense or antisense orientation. These transgenic fungi having enhanced γ-glutamyl transfer activity may be selected from the Ascomycetes class. The fungi may be selected from the Onygenaceae family, and in particular from the Histoplasma genus. In particular, the transgenic fungal species may be transgenic Histoplasma capsulatum.

The terms “fungus” or “fungi” include a variety of nucleated, spore bearing organisms which are devoid of chlorophyll. Examples include yeasts, mildews, molds, rusts, and mushrooms. Examples of fungi include, but are not limited to members of the genera Aspergillus, Candida, Cryptococcus, lssatchenkia, Coccidioides, Paracoccidioides, Histoplasma, Blastomyces, and Neurospora. In one embodiment, the fungi of the invention include fungi of the genus Histoplasma (e.g., Histoplasma capsulatum).

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication.

Provided are targets for fungicides, which include the gene products of nucleic acid sequences according to the present invention. For example, such targets may include nucleic acids from a fungus coding for a protein having the activity of a γ-glutamyltransferase. These nucleic acid sequences may include: a) a nucleic acid sequence having the nucleic acid sequence depicted in SEQ ID NO:1; or b) a nucleic acid sequence which can be derived by back-translation from the amino acid sequence depicted in SEQ ID NO:2, due to degeneracy of the genetic code; or c) functional equivalents of the nucleic acid sequences SEQ ID NO:1, which are at least 95% identical to SEQ ID NO:1. “Fungicides” are compounds that are used to kill or inhibit the growth of fungi or fungal spores.

Methods are provided for identifying substances with antifungal activity, where transcription, expression, translation, or activity of the gene products of the polynucleotides of the present invention is influenced. Those substances which reduce or block transcription, expression, translation or activity of the gene products are selected.

The term “antifungal activity” includes inhibiting the growth of a fungus (e.g., fungistatic activity), killing at least a portion of the fungus (e.g., fungicidal activity), limiting the ability of the fungus to reproduce, etc. The term “inhibiting the growth of a fungus” includes both fungistatic and fungicidal activity. Fungistatic activity includes any decrease in the rate of growth of a fungal colony. Fungistatic activity may be manifested by a fungus maintaining its present size or failing to colonize the surrounding areas. Fungistatic activity may be a result of inhibition of the fungal reproductive processes. Fungicidal activity generally includes, for example, eradication of a fungus or fungal colony, killing a fungus or fungal colony or, in one embodiment, a decrease in the mass or size of a fungus or fungal colony.

The nucleic acids of the present invention and their products may be used as targets for discovering antifungal substances. The inventive methods for identifying compounds with antifungal action comprise influencing transcription, expression, translation, and/or activity of the gene product of the nucleic acid sequence of the invention and selecting those compounds which reduce or block transcription, expression, translation and/or activity of the gene product, and the nucleic acid sequence of the invention is to be selected from the group consisting of the following sequences: a) a nucleic acid sequence with that in SEQ ID NO: 1; or b) a nucleic acid sequence which can be derived by back-translation of the amino acid sequence depicted in SEQ ID NO: 2, due to degeneracy of the genetic code; or c) a nucleic acid sequence which can be derived by retranslation of a functional equivalent of the amino acid sequence depicted in SEQ ID NO:2, due to degeneracy of the genetic code; or d) functional analogs of the nucleic acid sequence depicted in SEQ ID NO:1, which code for a polypeptide having the amino acid sequence depicted in SEQ ID NO: 2; or e) functional analogs of the nucleic acid sequence depicted in SEQ ID NO:1, which code for functional analogs of the amino acid sequence depicted in SEQ ID NO: 2.

Reduction of transcription, expression, translation and/or activity of the gene product means a reduction in the biological activity compared to the natural activity of the gene product by at least 10%, advantageously at least 20%, preferably at least 30%, particularly preferably at least 50% and very particularly preferably at least 70%. Blocking of the activity of the gene product means 100% blocking of the activity or partial blocking of the activity, preferably at least 80%, particularly preferably at least 90%, very particularly preferably at least 95%, blocking of the biological activity.

One embodiment of the methods for identifying compounds with antifungal action comprises the following steps: contacting a nucleic acid molecule of the present invention or a product of the nucleic acid molecule of the present invention (γ-glutamyltransferase) with one or more test compounds under conditions which allow binding of the test substance(s) to the nucleic acid molecule or to the γ-glutamyltransferase encoded by the aforementioned nucleic acid molecule; and: (i) detecting whether the test compound binds to the γ-glutamyltransferase, and/or (ii) detecting whether the test compound reduces or blocks the activity of the γ-glutamyltransferase, and/or (iii) detecting whether the test compound reduces or blocks transcription, translation or expression of the nucleic acid encoding γ-glutamyltransferase of the present invention.

The methods of the invention may be carried out in separate individual processes in vivo or in vitro and/or advantageously together or particularly advantageously in a high throughput screening and used for identifying compounds with antifungal action. These compounds may be inhibitors of the expression or and/or function of the genes and/or the proteins of the present invention.

When a sample to be tested which contains a compound with antifungal action, identified according to the methods of the invention, has been identified, then it is either possible to isolate this compound directly from the sample. Alternatively, it is possible to divide the sample into different groups, for example if it consists of a multiplicity of different test compounds, in order to reduce in this way the number of different test compounds per sample and then to repeat the method of the invention with such a “subsample”. Depending on the complexity of the sample, the above described steps may be repeated several times, preferably until the sample tested according to the method of the invention retains only a small number of compounds or only one compound with antifungal action.

High-throughput screening (HTS) makes possible parallel testing of a multiplicity of different compounds. When carrying out HTS, it is possible to use supports which may contain one or more of the nucleic acid molecules of the invention, one or more of the vectors containing the nucleic acid sequence of the invention, one or more transgenic organisms which contain at least one of the nucleic acid sequences of the invention or one or more (poly)peptides encoded via the nucleic acid sequences of the invention. The supports used may be solid or liquid, are preferably solid, particularly preferably microtiter plates. The aforementioned supports are likewise subject matters of the present invention. According to the most common technique, 96-well, 384-well or 1,536-well microtiter plates are used which usually can contain volumes of from 50 to 500 μl, preferably 200 μl. Apart from the microtiter plates, the other components of an HTS system which fit the particular microtiter plates, such as many instruments, materials, automated pipettors, robots, automated plate readers and plate-washing devices, are commercially available.

Apart from the HTS methods based on microtiter plates, it is also possible to use “free format assays” or assay systems which have no physical barriers between the samples, as, for example, in Jayawickreme et al., 1994, Proc. Natl. Acad. Sci. USA 91: 1614-1618; and U.S. Pat. No. 5,976,813 (“Continuous format high throughput screening”).

One embodiment of the in vitro method comprises the steps in which a) either the polypeptide is expressed in enzymatically active form in a transgenic organism of the invention or an organism containing the protein of the invention is cultured; b) the protein obtained in step a) is incubated with a test compound directly in the resting or growing organism, in the cell extract of the transgenic organism, in a partially purified form or in a form purified to homogeneity; c) a test compound is selected by step b), which inhibits a polypeptide encoded by a nucleic acid sequence of the invention. The term “inhibits” is to be treated as equivalent to the term “significant reduction in enzymatic activity.”

An analogous form of the above method comprises a) either expressing γ-glutamyltransferase in a transgenic organism or culturing an organism which naturally contains γ-glutamyltransferase encoded by a nucleic acid sequence of the invention; b) contacting γ-glutamyltransferase from the organism of step a) in the cell extract of the organism, in a partially purified form or in a form purified to homogeneity, with a test compound; and c) selecting a test compound which reduces or blocks the γ-glutamyltransferase activity, the activity of the γ-glutamyltransferase incubated with a test compound being determined using the activity of a γ-glutamyltransferase not incubated with a test compound. To this end, compounds which result in a significant decrease in enzymatic activity are selected in both of the above method variants in step (c), achieving a reduction of at least 10%, advantageously at least 20%, preferably at least 30%, particularly preferably at least 50% and very particularly preferably at least 70%, or 100% reduction (blocking). In a preferred embodiment, the compounds with antifungal action or active compounds are identified by determining the enzymatic activity in the presence and absence of a compound to be studied.

The γ-glutamyltransferase which is required for the assay may be isolated either from a transgenic organism of the invention by means of heterologous expression or from an organism containing γ-glutamyltransferase, for example from a fungus, preferably from one of the Histoplasma genus, and more preferably from Histoplasma capsulatum.

The solution containing the polypeptides of the present invention may comprise the lysate of the original organism or the transgenic organism. Alternatively, the polypeptides of the present invention may be isolated from culture supernatant. If necessary, the polypeptides of the invention can be purified partially or completely via common methods. A general overview of common techniques for purifying proteins is given, for example, in Ausubel et al., 1993. In the case of recombinant preparation, the protein fused to an affinity tag may be purified via affinity chromatography.

The enzymatic activity is determined by incubating the polypeptides of the invention with a suitable substrate, and substrate conversion or increase in the resulting product is monitored. An example of a suitable substrate is γ-glutamyl-p-nitroanilide (GpNA) as the donor of the γ-glutamyl residue. In a transpeptidation-type reaction, glycylglycine may be used as an acceptor substrate. Preference is given here to substrates whose decrease or increase can be monitored spectrophotometrically. In a particularly preferred embodiment, substrate conversion is spectrophotometrically monitored, following a method described by Strassman et al., 1966, J. Biol. Chem. 241: 5401-5407.

Methods of detecting GGT polynucleotides in a sample are provided as well. This may be achieved by first contacting the sample with a GGT polynucleotide of the present invention or a complement thereof, or contacting the sample with a polynucleotide that comprises a sequence of at least 12 nucleotides and is complementary to a contiguous sequence of a GGT polynucleotide of the present invention. Then, it is determined whether a hybridization complex has been formed. In one example, the at least 12 nucleotide sequence will comprise a domain conserved among γ-glutamyltransferase genes.

Expression regulatory elements are also provided, and in particular a native promoter or variant of a native promoter from Histoplasma capsulatum that can be used to express the genes (e.g., SEQ ID NO:1) and proteins (e.g., SEQ ID NO:2) of the present invention. In one embodiment, the promoter is a native promoter from Histoplasma capsulatum that controls expression of the γ-glutamyltransferase gene.

A “nucleic acid probe” or “oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. For example, probes may be peptide nucleic acids in which the constituent bases are joined by peptide bonds rather than phosphodiester linkages. It will be understood that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

“Increased or enhanced expression or activity of a polypeptide of the present invention,” or “increased or enhanced expression or activity of a polynucleotide encoding a polypeptide of the present invention,” refers to an augmented change in activity of the polypeptide or protein. Examples of such increased activity or expression include the following: (1) activity of the protein or expression of the gene encoding the protein is increased above the level of that in wild-type, non-transgenic control organisms; (2) activity of the protein or expression of the gene encoding the protein is in an organ, tissue or cell where it is not normally detected in wild-type, non-transgenic control organisms (i.e., spatial distribution of the protein or expression of the gene encoding the protein is altered); (3) activity of the protein or expression of the gene encoding the protein is increased when activity of the protein or expression of the gene encoding the protein is present in an organ, tissue or cell for a longer period than in a wild-type, non-transgenic controls (i.e., duration of activity of the protein or expression of the gene encoding the protein is increased). For example, increased activity or expression of a gene encoding a protein of the present invention above the level of that in comparable wild-type, non-transgenic control organism refers to increased activity or expression by at least 10%, advantageously at least 20%, preferably at least 30%, particularly preferably at least 50% and very particularly preferably at least 70%.

“Decreased expression or activity of a protein or polypeptide of the present invention,” or “decreased expression or activity of a nucleic acid or polynucleotide encoding a protein of the present invention,” refers to a decrease in activity of the protein. An example of such decreased activity or expression includes the decrease in activity of the protein or expression of the gene encoding the protein below the level of that in wild-type, non-transgenic control fungi.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

In the case of both expression of transgenes and inhibition of endogenous genes (e.g., by antisense, or sense suppression) the inserted polynucleotide sequence need not be identical and may be “substantially identical” to a sequence of the gene from which it was derived. As explained below, these variants are specifically covered by this term.

In the case where the inserted polynucleotide sequence is transcribed and translated to produce a functional polypeptide, because of codon degeneracy, a number of polynucleotide sequences will encode the same polypeptide. These variants are specifically covered by the term “polynucleotide sequence from” a particular gene. In addition, the term specifically includes sequences (e.g., full length sequences) substantially identical (determined as described below) with a gene sequence encoding a protein of the present invention and that encode proteins or functional fragments that retain the function of a protein of the present invention, e.g., γ-glutamyl transfer activity as well as iron reduction activity in the presence of glutathione.

In the case of polynucleotides used to inhibit expression of an endogenous gene, the introduced sequence need not be perfectly identical to a sequence of the target endogenous gene. The introduced polynucleotide sequence will typically be at least substantially identical (as determined below) to the target endogenous sequence.

Optimal alignment of sequences for comparison may be conducted by methods commonly known in the art, e.g., by the search for similarity method (Pearson and Lipman 1988, Proc. Natl. Acad. Sci. USA 85: 2444-2448), by computerized implementations of algorithms such as GAP, BESTFIT, BLAST, FASTA, and TFASTA in the Wisconsin Genetics Software Package, Genetics Computer Group (GCG), Madison, Wis., or by inspection.

In a preferred embodiment, protein and nucleic acid sequence identities are evaluated using the Basic Local Alignment Search Tool (“BLAST”) which is well known in the art (Karlin and Altschul, 1990, Proc. Natl. Acad. Sci. USA 87: 2267-2268; Altschul et al., 1997, Nucl. Acids Res. 25: 3389-3402) the disclosures of which are incorporated by reference in their entireties. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990). The BLAST programs can be used with the default parameters or with modified parameters provided by the user.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. These values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99%. Polypeptides that are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine. Accordingly, polynucleotides of the present invention encoding a protein of the present invention include nucleic acid sequences that have substantial identity to the nucleic acid sequence of SEQ ID NO:1. Polypeptides or proteins of the present invention include amino acid sequences that have substantial identity to SEQ ID NO:2.

In one aspect, the invention also relates to nucleic acids that selectively hybridize to the exemplified sequences, including hybridizing to the exact complements of these sequences. The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide, which decreases DNA duplex stability. In general, the longer the probe, the higher the temperature required for proper annealing. A common approach is to vary the temperature: higher relative temperatures result in more stringent reaction conditions.

To hybridize under “stringent conditions” describes hybridization protocols in which nucleotide sequences at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

“Stringent hybridization conditions” are conditions that enable a probe, primer or oligonucleotide to hybridize only to its target sequence. Stringent conditions are sequence-dependent and will differ. Stringent conditions comprise: (1) low ionic strength and high temperature washes, e.g. 15 mM sodium chloride, 1.5 mM sodium citrate, 0.1% sodium dodecyl sulfate at 50° C.; (2) a denaturing agent during hybridization, e.g. 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate) at 42° C.; or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% homologous to each other typically remain hybridized to each other. These conditions are presented as examples and are not meant to be limiting.

“Moderately stringent conditions” use washing solutions and hybridization conditions that are less stringent (Sambrook et al., 1989), such that a polynucleotide will hybridize to the entire, fragments, derivatives, or analogs of SEQ ID NO:1. One example comprises hybridization in 6×SSC, 5×Denhardt's solution, 0.5% SDS and 100 μg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions have been described (Ausubel et al., 1993; Kriegler, 1990).

“Low stringent conditions” use washing solutions and hybridization conditions that are less stringent than those for moderate stringency (Sambrook et al., 1989), such that a polynucleotide will hybridize to the entire, fragments, derivatives, or analogs of SEQ ID NO:1. A nonlimiting example of low stringency hybridization conditions are hybridization in 35% formamide, 5×SSC, 50 mM Tris HCl (pH 7.5), 5 mM EDTA, 0.02% PVP, 0.02% Ficoll, 0.2% BSA, 100 μg/ml denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency, such as those for cross species hybridizations are well-described (Ausubel et al., 1993; Kriegler, 1990).

The nucleic acids and proteins of the present invention may be isolated using methods known in the art. The genes or nucleic acid sequences encoding proteins of the present invention includes genes and gene products identified and characterized by analysis using the nucleic acid sequences, including SEQ ID NO:1 and protein sequences, including SEQ ID NO:2. Sequences encoding proteins of the present invention include nucleic acid sequences having substantial identity to SEQ ID NO:1. Polypeptides of the present invention include polypeptides having substantial identity to SEQ ID NO:2. Preferred nucleic acids of the present invention encode proteins involved in γ-glutamyl transfer activity as well as iron reduction activity in the presence of glutathione.

The isolation of sequences from the genes used in the methods of the present invention may be accomplished by a number of techniques. For instance, oligonucleotide probes based on the sequences disclosed here can be used to identify the desired gene in a cDNA or genomic DNA library from a desired fungal species. To construct genomic libraries, large segments of genomic DNA are generated by random fragmentation, e.g. using restriction endonucleases, and are ligated with vector DNA to form concatemers that can be packaged into the appropriate vector.

The cDNA or genomic library can then be screened using a probe based upon the sequence of a cloned gene such as the polynucleotides disclosed here. Probes may be used to hybridize with genomic DNA or cDNA sequences to isolate homologous genes in the same or different fungal species.

Alternatively, the nucleic acids of interest can be amplified from nucleic acid samples using amplification techniques. For instance, polymerase chain reaction (PCR) technology can be used to amplify the sequences of the genes directly from mRNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes.

Appropriate primers and probes for identifying genes encoding a protein of the present invention can be generated from comparisons of the sequences provided herein. For a general overview of PCR see PCR Protocols, 2003, Bartlett and Stirling, eds., 2^{nd} edition, Humana Press, which is herein incorporated by reference. For examples of primers used see examples section below.

Polynucleotides may also be synthesized by well-known techniques as described in the technical literature (Adams et al., 1983, J. Am. Chem. Soc. 105: 661-663). Double stranded DNA fragments may then be obtained either by synthesizing the complementary strand and annealing the strands together under appropriate conditions, or by adding the complementary strand using DNA polymerase with an appropriate primer sequence.

One useful method to produce the nucleic acids of the present invention is to isolate and modify the nucleic acid sequences of the present invention. This can be done using methods of sequence-specific mutagenesis of a nucleic acid, e.g., oligonucleotide-directed mutagenesis as well as directed mutagenesis of nucleic acids using PCR. Such methods are useful to insert specific codon changes in the nucleic acids of the invention.

Once a nucleic acid is isolated using the method described above, standard methods can be used to determine if the nucleic acid is a preferred nucleic acid of the present invention and therefore encodes a preferred protein of the present invention, e.g., by using structural and functional assays known in the art. For example, the sequence of a putative nucleic acid sequence thought to encode a preferred protein of the present invention can be compared to a nucleic acid sequence encoding a preferred protein of the present invention to determine if the putative nucleic acid is a preferred polynucleotide of the present invention.

Enhancing/increasing or decreasing expression of a gene of the present invention in a fungus may modulate iron reduction processes by a variety of pathways. The particular pathway used to modulate iron reduction is not critical to the present invention.

The polypeptides encoded by the genes of the invention, like other proteins, have different domains which perform different functions. Thus, the gene sequences need not be full length, so long as the desired functional domain of the protein is expressed.

Vectors useful for practicing the present invention can be prepared using methods known in the art. Typical vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. A catalogue of bacteria and bacteriophages useful for cloning is provided, e.g., by the ATCC, e.g., The ATCC Catalogue of Bacteria and Bacteriophages, 1992, Gherna et al., eds., published by the ATCC. Additional procedures for sequencing, cloning and other aspects of molecular biology and underlying theoretical considerations are also found in Watson et al., 1992, Recombinant DNA, 2^{nd} edition, W.H. Freeman & Co., New York, N.Y., which is herein incorporated by reference.

To use isolated sequences in the above techniques, recombinant DNA vectors suitable for transformation of desired organisms are typically prepared. For example, techniques for genetic manipulation in fungi and for transforming a wide variety of fungal species are well known and described in the scientific literature. Transformation and expression in filamentous fungi may involve homologous expression (see, for example, Stohl et al., 1983, Proc. Natl. Acad. Sci. USA 80: 1058-1062; Grant et al., 1984, Mol. Cell. Biol. 4: 2041-2051; for a review of homologous transformation see Hynes, 1986, J. Exper. Mycology 10:1-8). U.S. Pat. No. 6,255,115 (“Agrobacterium mediated transformation of moulds, in particular those belonging to the genus Aspergillus”) discloses the transformation of filamentous fungi with homologous genes using the Agrobacterium tumefaciens Ti plasmid, affording a method of producing recombinant mold strains free of bacterial DNA contamination. Examples of heterologous expression in fungi are described in U.S. Pat. No. 5,814,495 (“Melanin production by Streptomyces). Vectors for expression and secretion of heterologous proteins in filamentous fungi, devoid of bacterial DNA, are described in U.S. Pat. No. 6,171,817 (“Heterologous polypeptides expressed in filamentous fungi, process for making same, and vectors for making same”). U.S. Pat. No. 6,090,574 (“Process for preparing a protein by a fungus transformed by multicopy integration of an expression vector”) discloses fungal transformation with multicopy integration, which results in an increased production of the desired protein. U.S. Pat. No. 6,403,362 (“Systems for the mass production of proteins or peptides by microorganisms of the genus humicola”) discloses expressing systems which enable a large amount of production of a protein. All of the above publications and patents are herein incorporated by reference.

To use isolated sequences in the above techniques, recombinant DNA vectors suitable for transformation of desired organisms are prepared. A DNA sequence coding for the desired polypeptide, for example a cDNA sequence encoding a full length protein, will preferably be combined with transcriptional and translational initiation regulatory sequences that will direct the transcription of the sequence from the gene in the intended cells or tissues of the transformed organism.

The vector comprising the sequences (e.g., promoters or coding regions) from genes of the invention may comprise a marker gene that confers a selectable phenotype on transformed organisms. For example, the marker may encode biocide resistance, particularly antibiotic resistance, such as resistance to kanamycin, G418, bleomycin, or hygromycin. In some instances of recombinant polypeptide expression, a polyadenylation region at the 3′-end of the coding region should be included. The polyadenylation region can be derived from the natural gene, from a variety of other fungal genes, or from T-DNA.

Nucleic acid sequences of the present invention can be expressed recombinantly in organisms to modulate γ-glutamyltransferase activity and/or iron reduction. A variety of different expression constructs, such as expression cassettes and vectors suitable for transformation of desired organisms can be prepared. A DNA sequence coding for a polypeptide described in the present invention can be combined, for example, with cis-acting (promoter and enhancer) transcriptional regulatory sequences to direct the timing, tissue type and levels of transcription in the intended tissues of the transformed organism. Translational control elements can also be used.

In one aspect, the invention provides a nucleic acid operably linked to a promoter which, in some embodiments, is capable of driving the transcription of the coding sequence in transgenic organisms. The promoter can be, e.g., derived from fungal sources. The promoter can be, e.g., constitutively active, inducible, or tissue specific. In construction of recombinant expression cassettes, vectors, transgenics, of the invention, different promoters can be chosen and employed to differentially direct gene expression, e.g., in some or all cells, or in an inducible manner. Typically, desired promoters are identified by analyzing the 5′ sequences of a genomic clone corresponding to the genes described.

In another aspect, the present invention provides expression cassettes or vectors, host cells, or transgenic organisms comprising expression cassettes or vectors comprising a Histoplasma capsulatum promoter operably linked to a nucleic acid of the present invention. The promoters and nucleic acids can be operably linked using standard recombinant techniques. The promoter may be homologous or heterologous to the nucleic acid.

In some embodiments, transgenic fungal lines can be generated with silent expression cassettes in which desired genes are introduced either in sense or antisense orientation under an inducible promoter that is activated by application of an external inducer, when the fungus reaches a certain developmental stage, or when it is grown under specific environmental conditions. Activation of the promoter will lead to production of a protein or RNA molecule of the present invention.

In some embodiments, a promoter fragment can be employed which will direct expression of a nucleic acid of the present invention in all transformed cells or tissues f an organism. Such promoters are referred to herein as “constitutive” promoters and are active under most environmental conditions and states of development or cell differentiation. Examples of constitutive promoters include those from viruses, such as the cauliflower mosaic virus (CaMV) 35S transcription initiation region (Dagless, 1997, Arch. Virol. 142: 183-191); the 1′- or 2′-promoter derived from T-DNA of Agrobacterium tumefaciens (O'Grady, 1995, Plant Mol. Biol. 29: 99-108); the promoter of the tobacco mosaic virus; the promoter of Figwort mosaic virus (Maiti, 1997, Transgenic Res. 6: 143-156); and actin promoters, such as the Arabidopsis actin gene promoter (Huang, 1997, Plant Mol. Biol. 33: 125-139).

In one aspect, the present invention provides native promoters from Histoplasma capsulatum. In particular, the present invention provides γ-glutamyltransferase promoters from Histoplasma capsulatum capable of controlling expression of the genes of the present invention. A γ-glutamyltransferase promoter from Histoplasma capsulatum is a promoter derived from a Histoplasma capsulatum γ-glutamyltransferase gene, e.g., by cloning, isolating or modifying a native promoter from a γ-glutamyltransferase. The provided promoters can be used to initiate gene expression in transgenic organisms, e.g. in transgenic fungi.

Preferred promoters of the present invention can control expression of the GGT gene. Accordingly, the preferred promoters can control expression of genes comprising coding regions that have substantial identity to the coding region of SEQ ID NO:1, e.g., preferably at least 70%, at least 80%, at least 90%, or at least 95%, 95%, 97%, 98%, 99%, or 100% identity to the coding regions of SEQ ID NO:1.

A variety of promoters capable of expressing an operablγ-linked coding sequence in a fungal host may be used. These promoters may be isolated from various prokaryotes or eukaryotes.

Examples of fungal promoters suitable for the practice of the present invention include: fungal promoters active in the presence of glucose (U.S. Pat. No. 6,011,147); fungal cutinase promoter that is inducible by a plant component (Bajar et al., 1991, Proc. Natl. Acad. Sci. USA 88: 8208-8212). Examples of other suitable promoters; constitutive, inducible, etc., can be found in Arora, 2003, Handbook of Fungal Technology, 2^{nd} ed., Marcel Dekker; and in Tkacz and Lange, 2004, Advances in Fungal Biotechnology for Industry, Agriculture, and Medicine, Springer. U.S. Patent Application Publication No. US 2006/0040342 A1 discloses isolated fungal promoters and gene transcription terminators and methods of protein and chemical production in a fungus.

Transgenic organisms of the present invention can be prepared using methods known in the art. DNA constructs of the invention may be introduced into the genome of a desired host by a variety of conventional techniques. For example, the DNA construct may be introduced directly into the genomic DNA of the host cell using techniques such as electroporation and microinjection, or the DNA constructs can be introduced directly to the cells using biolistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the host cell DNA when the cell is infected by the bacteria.

Using known procedures, screens for fungi of the invention can be performed by detecting increased or decreased levels of the claimed gene and claimed protein in a fungus and detecting the desired phenotype. Means for detecting and quantifying mRNA or proteins are well known in the art, e.g., Northern Blots, RT-PCR, DNA microarrays, Western Blots or protein activity assays.

Gene amplification and/or expression can be measured in a sample directly, for example, by conventional Southern blotting, Northern blotting to quantitate the transcription of mRNA, dot blotting (DNA analysis), DNA microarrays, or in situ hybridization, using an appropriately labeled probe, based on the sequences provided herein. Various labels can be employed, most commonly radioisotopes, particularly ³²P. However, other techniques can also be employed, such as using biotin-modified nucleotides for introduction into a polynucleotide. The biotin then serves as the site for binding to avidin or antibodies, which can be labeled with a wide variety of labels, such as radionuclides, fluorescers, enzymes, or the like. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. Gene expression can also be measured using DNA microarrays, commonly known as gene chips.

In one aspect, the present invention also provides for antibodies immunologically specific for all or part, e.g., an amino-terminal portion, of a polypeptide at least 70% identical to a sequence as shown in SEQ ID NO:2. The antibodies may be immunologically specific for all or part, e.g., an amino-terminal portion, of a GGT polypeptide encoded by an isolated nucleic acid which hybridizes under stringent conditions to a sequence as shown in SEQ ID NO:1 or the complement thereof. Accordingly, the present invention provides isolated antibody or antibody compositions that specifically bind to a polypeptide having the amino acid sequence as shown in SEQ ID NO:2. In some embodiments, the antibody is monoclonal. In other embodiments, the antibody is polyclonal. In some examples, the antibodies of the present invention are labeled.

The invention further provides methods of detecting GGT polypeptides in a sample, by way of contacting the sample with an anti-GGT antibody of the present invention, and subsequently determining whether a hybridization complex has been formed between the antibody and the polypeptide.

The polypeptides of the present invention may be used alone or in combination with other proteins or agents to modulate γ-glutamyltransferase activity and/or to modulate iron reduction.

The ferric reductase/GGT enzyme and/or the peptide ferric reductant it generates could serve as antifungal antibiotic targets, for Histoplasma capsulatum or for other fungi or other microbes. In one aspect of the invention, GGT inhibitors are provided that can inhibit growth of the Histoplasma, demonstrating the potential value of GGT or the dipeptide Cys-Gly as drug targets.

### Examples

Fungal strains and growth conditions. The G217B strain (ATCC 26032, American Type Culture Collection, Manassas, Va.) of H. capsulatum var. capsulatum Darling was used. Unless specified, the fungus was maintained in a rich defined Histoplasma-macrophage medium broth (HMM) (Worsham and Goldman, 1988, J. Med. Vet. Mycol. 26: 137-143). All cells were grown in a 5% CO₂-95% air atmosphere. For growth curve analysis, yeast cells were taken from late-log-phase cultures and resuspended at a concentration of 3×10⁵ cells/ml in 20 ml of HMM (A₆₀₀ of 1 corresponds to 2.24×10⁸ CFU/ml). Culture turbidity was monitored with a Klett-Summerson photoelectric colorimeter (Manostat Corporation, New York, N.Y.).

Purification of GSH-FeR. 1.5 l batch fungal cultures were grown in 2.5 l Erlenmeyer flasks in Histoplasma capsulatum Minimal Medium (HcMM) broth for 5 days (Zarnowski et al., 2008, Curr. Microbiol. 56: 110-114). The supernatant (total of 18 l) was filter-sterilized and concentrated down to about 100 ml using a Vivaflow 200 unit (Sartorius AG, Goettingen, Germany) equipped with a hydrosart 30-kDa cut-off membrane. A final concentration of high-molecular-mass fractions to about 20 ml was achieved using Vivaspin 20 units equipped with polyethersulfone 5-kDa nominal molecular mass cut-off limit membranes (Sartorius). All chromatographic separation steps were performed at room temperature on the high-performance liquid chromatography ÄKTA-Purifier 10 system (Amersham Biosciences AB, Uppsala, Sweden). All buffers used were filtered through 0.2 μm nylon membrane filters (Nalgene, Rochester, N.Y.). Protein amounts were assessed using the BCA Protein Assay Kit (Pierce Biotechnology, Rockford, Ill.), with bovine serum albumin as a standard. The sample was chromatographically desalted on a HiPrep™26/10 Desalting column (Amersham) and then separated on an anion exchanger HiPrep™16/10 DEAE FF column (Amersham) equilibrated with 20 mM Tris/HCl (pH 8.0). Elution was carried out in a 20 mM Tris/HCl (pH 8.0)/1.0 M NaCl buffer system and proteins were eluted at a flow rate of 1 ml min⁻¹ in a linear gradient of salt from 0 to 30% in 25 column volumes. GSH-FeR positive fractions were pooled together, concentrated and applied to gel filtration on a HighPrep 16/60 Sephacryl™ S-300 HR column (Amersham) equilibrated with PBS. Proteins were eluted at a flow rate of 0.5 ml min⁻¹ and 1 ml fractions were collected. Fractions containing GSH-FeR activity were concentrated and desalted on the HiPrep™26/10 Desalting column, and subsequently separated in anion exchange chromatography on a MonoQ™ 5/50 GL column (Amersham) equilibrated with 20 mM bis-Tris/HCl (pH 6.5). Proteins were eluted at a flow rate of 1 ml min⁻¹ in the same buffer containing 0.5 M NaCl in an extended linear gradient from 0 to 45% in 90 column volumes. GSH-FeR-positive fractions were collected, concentrated and separated in gel filtration chromatography on two Superdex™ 200 10/300 GL columns (Amersham) set in a row. The columns were preequilibrated with PBS and proteins were eluted in 2.2 column volumes of this buffer at a flow rate of 0.5 ml min⁻¹. Fractions containing the purified protein were filter-concentrated and resuspended in a small volume of Protein Stabilizing Cocktail (Pierce). The enzyme was stable for at least 6 months when stored at 4° C.

In addition, a pool of purified GSH-FeR was subjected to chromatofocusing on a MonoP™ 5/200 GL column (Amersham). Proteins were separated in a linear pH gradient in a range of 7-4 in two buffer systems: 25 mM bis-Tris, pH 7.1 was utilized as a starting buffer, and 10% (v/v) Polybuffer 74, pH 4.0 was applied in a protein elution step. Both buffers were adjusted to desired pH values with a saturated solution of iminodiacetic acid.

Internal amino acid sequencing. The trypsin digestion procedure used is a modified version of the protocol published elsewhere (Shevchenko et al., 1996, Anal. Chem. 68: 850-858). GSH-FeR-positive fractions transferred into a 1.5-ml Eppendorf tube were reduced with 150 μl of 100 mM NH₄CO₃ and 10 μl of 45 mM DTT for 30 min at 60° C., and subsequently alkylated with 10 μl of 100 mM iodoacetic acid for 20 min. The solvent was removed in a rotary evaporator and the dried samples were rehydrated with a digestion solution consisting of 30 μl of 100 mM NH₄HCO₃ containing 0.3 μg sequencing grade modified trypsin (Promega, Madison, Wis.). The reaction was carried out at room temperature overnight and subsequently subjected to nanospray LC-tandem MS analysis. Samples were loaded onto a monolithic in-house fabricated capillary column (Polymicro Technologies, Phoenix, Ariz.) packed with Aquapore C₁₈ media (Applied Biosystems, Inc., Foster City, Calif.). All mass spectra were obtained on a Finnigan LCQ Deca XP Ion Trap mass spectrometer (ThermoFinnigan, San Jose, Calif.). Data were acquired in a data-dependent manner (dynamic exclusion repeat count of 2 and repeat duration of 1 min) using a triple play method, in which a full scan was followed by a zoom scan and tandem MS of the most abundant ion in that scan. In the absence of a completed database for H. capsulatum, all tandem MS spectra were manually examined and interpreted for sequence information. In some cases, derived sequences were searched for similarity profiles with related fungal genomes using BLAST (Basic Local Alignment Search Tool).

Enzymatic assays. GGT activity of the purified enzyme was determined in a microtitre plate-based assay using a synthetic substrate GpNA as the donor of the γ-glutamyl residue. In the transpeptidation-type reaction, 20 mM glycylglycine was used as an acceptor substrate. The total reaction volume was 150 μl. The reaction was carried out in PBS (except where noted) at 37° C. and then amounts of released p-nitroanilide were determined spectrophotometrically at 412 nm. To determine kinetics parameters of Ggt1-catalyzed GpNA decomposition, the substrate was used at concentrations up to 2.67 mM. The Kₘ and Vₘₐₓ values with their standard deviations were calculated for all the enzyme assays by a non-linear least squares regression fit of the measured (v, [S]) values to a hyperbola using the PRISM software (GraphPad Software, San Diego, Calif.). Standard deviations calculated were in general less than 8% of derived parameter values, indicating a good fit of the data to the Michaelis-Menten relationship. To determine the effect of pH upon Ggt1 activity, 150 mM MES (pH 5.5-6.5) and 150 mM HEPES (pH 7.0-8.5) were used.

Iron reduction activity was determined with GSH or cysteinylglycine in a microtitre plate-based assay as described elsewhere (Zarnowski and Woods, 2005, Microbiology-SGM 151: 2233-2240). Total iron reduction activity of the studied system was determined with Ggt1 and GSH added, and ferric nitrate chelated with an equal molar ratio of nitrilotriacetic acid was used as a substrate. The formation of ferrous ions was quantified with the chromogenic chelator ferrozine (Sigma). The total reaction volume was 120 μl. The reaction was carried out in PBS (except where noted) at 37° C. and then the absorbance was measured at 562 nm. The negative control contained all appropriate compounds and no non-enzymatic iron reduction was observed under the conditions described above. To determine the effect of pH upon iron reduction activity, 150 mM MES (pH 5.5-6.5) and 150 mM HEPES (pH 7.0-8.5) were used. To establish the role of thiol group in iron reduction process, GSH and its derivatives such as glutathione ethyl ester (GSHEt), oxidized glutathione (GSSG), S-methylglutathione (GSMe), were tested at 1 mM doses.

Ggt1-catalyzed GSH hydrolysis ratios as a function of pH were calculated as follows: Firstly, the total iron reduction capability of this reaction system with both Ggt1 and GSH added as described above was measured. Then, Kₘ and Vₘₐₓ values with their standard deviations for cysteinylglycine-catalyzed iron reduction in different buffer systems using a PRISM-assisted non-linear regression (GraphPad Software) were determined. This step enabled the calculation of amounts of cysteinylglycine released from GSH under various pH conditions. As molar ratios of the dipeptide release and GSH decomposition are equal in this reaction, it was possible to determine the extent of Ggt1-catalyzed GSH hydrolysis.

0.1 mM 6-diazo-5-oxo-L-norleucine (DON), serine/borate complex (SBC), and acivicin were tested as classical GGT activity inhibitors (Tate and Meister, 1981, Mol. Cell. Biochem. 39: 357-368; Tate and Meister, 1981, Proc. Natl. Acad. Sci. USA 75: 4806-4809), whereas 0.1 mM aluminum and gallium (as nitrate salts) were examined as iron reductase activity inhibitors (Zarnowski and Woods, 2005). Prior to each reaction, the reaction mix was pretreated with individual inhibitors for 15 min, after which inhibitors were removed by diafiltration and proper substrates were added.

ESI-MS analysis. Analyses of products obtained after a Ggt1-catalyzed reaction were carried out on a 3200 QTRAP tandem mass spectrometer (Applied Biosystems, Foster City, Calif.) equipped with a Turbo V™spray source. The sample was injected as a 10 μl aliquot at 30 μl ml⁻¹ from the 4.6-mm diameter auto-syringe delivery system (Harvard Apparatus, Holliston, Mass.) in 50% MeOH. The following instrumental parameters were used to generate the most optimum protonated ions [M+H^{⊕}] in Positive Mode: ion spray voltage (1S), 5.5 kV; curtain gas (CUR), 20 psi; Nebulizer gas (GS1), 20 psi; turbo gas (GS2), 10 psi; turbo gas temperature (TEM), 150° C.; interface heater (ihe), ON; declustering potential (DP), 15 eV; entrance potential (EP), 10 eV; detector (CEM), 1,900; dwell time, 2,000 msec; pause time, 5.0 msec. Acquired data was processed using Analyst 1.4.2 software (Applied Biosystems) to deconvolute parent masses observed in the range from 100 to 1000 atomic mass units (amu).

Genetic manipulations. Plasmids were cloned and propagated in the Escherichia coli strain JM109. Vectors were isolated from E. coli by using an alkaline lysis QIAprep8 miniprep kit procedure according to the recommendations of the kit manufacturer (QIAGEN, Valencia, Calif.). DNA from agarose gels was purified by using the QIAquick silica gel extraction kit (QIAGEN). DNA was isolated from H. capsulatum by using a MasterPure yeast DNA purification kit according to the directions of the manufacturer (Epicentre Biotechnologies, Madison, Wis.). RNA was isolated from yeast cells using a RiboPure-Yeast kit (Ambion) and Superscript III reverse transcriptase (Invitrogen) was used in the synthesis of first-strand cDNA according to the supplier's protocol. All PCR products were amplified using the high-fidelity Triplemaster polymerase (Eppendorf, Westbury, N.Y.).

Plasmid pLBZ1 is an H. capsulatum expression vector derived from pWU55 (Woods et al., 1998, J. Bacteriol. 180: 5135-5143). It carries a PaURA5 marker for selection, an inverted telomeric region for linearization and maintenance, and Ascl and Sbfl cloning sites between the H2B 5′ and CATB 3′ flanking sequences. Primers were designed to amplify 686 by upstream of the H2B start codon, and 727 by downstream of the CATB stop codon from the G217B strain of H. capsulatum. The two products were then mixed together and used as a template for splice by overlap extension in a PCR reaction (SOE PCR). Ascl and Sbfl sites were included on the two internal primers to create cloning sites between the H2B and CATB regulatory sequences. BamHI sites were included on the two external primers, and the final SOE PCR product was cloned into the BamHI site of pWU55. This plasmid was used in further genetic manipulations of GGT1 overexpression and RNAi-mediated GGT1 silencing.

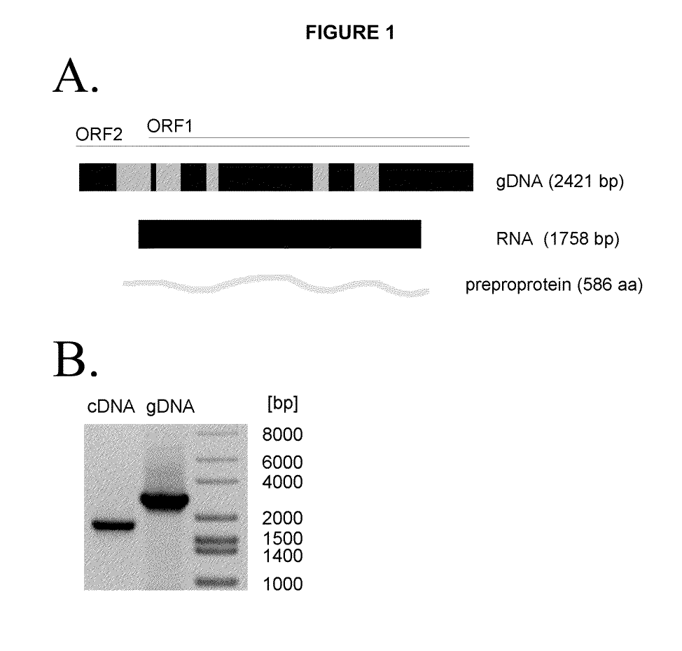

FIG. 1 is a graphical presentation of a gene encoding secreted γ-glutamyltransferase (GGT1) of H. capsulatum. FIG. 1(A): A genomic DNA sequence of GGT1 consists of 2421 by organized in 6 exons (in black) separated by five noncoding introns (in gray), which yield a total transcript of 1758 by (solid black bar) and a corresponding 586-aa preproprotein (light gray bar). Solid black lines labeled as “ORF1” and “ORF2” refer to two predicted GGT1 open reading frames. FIG. 1(B): PCR amplification of GGT1 from genomic and cDNA templates. The gene was amplified with a pair of GGT1.Ascl.ORF2.F and GGT1.Sbfl.ORF1.R primers.

For GGT1 overexpression, two putative GGT1 ORF sequences (1518-bp ORF1 and 1758-bp ORF2 shown in FIG. 1A) were amplified with two pairs of primers (GGT1.Ascl.ORF1.F and GGT1.Sbfl.ORF1.R for ORF1, and GGT1.Ascl.ORF2.F and GGT1.Sbfl.ORF1.R for ORF2, respectively) and subsequently cloned into the pLBZ1 plasmid. This step yielded two vectors, pLBZ1_OE::GGT1.ORF1 and pLBZ1_OE::GGT1.ORF2, respectively, which were subsequently electroporated into a uracil-auxotrophic H. capsulatum G217Bura5-23 strain (Woods et al., 1998) as described below.

For GGT1 RNAi silencing, 1502-bp GGT1 fragments were PCR amplified from cDNA and cloned in opposite orientations into pBluescript SK(+) (Stratagene, La Jolla, Calif.). The first fragment was amplified with GGT1.RNAi.Kpnl.Ascl.F1 and GGT1.RNAi.HindIII.R1 primers and cloned into Kpnl and HindIII restriction sites. The second fragment was amplified with GGT1.RNAi.Notl.Sbfl.F2 and GGT1.RNAi.Xbal.R2 primers and then cloned in opposite orientation into Notl and Xbal restriction sites. Ascl and Sbfl sites were also introduced with these primers. Extra loop sequence of 151 by amplified from the tetR gene on pWU55 using a pair of t-1 and t-2 primers was cloned into the BamHI site for a total loop sequence of 220 bp. The entire GGT hairpin was excised then from the resulting plasmid pBS_GGT1 RNAi and cloned into the H. capsulatum expression vector pLBZ1 yielding the pLBZ1_GGT1 RNAi plasmid.

The G217B ura5-23 strain was electrotransformed with both OE and RNAi-type vectors as previously described (Woods et al., 1998). Briefly, cells were grown for 42 h with shaking at 37° C., washed once with 10% mannitol, and electroporated with Pmel-digested, ethanol-precipitated pLBZ1_OE::GGT1.ORF1, pLBZ1_OE::GGT1.ORF2, pLBZ1_GGT1 RNAi or pLBZ1 as an empty-vector control in a Gene Pulser electroporator (Bio-Rad, Hercules, Calif.). Following transformation, cells were spread onto HMM plates and grown for 2 to 3 weeks at 37° C. Following growth on plates, individual colonies were selected and grown in liquid medium. Three-day liquid cultures of 72 transformants were screened and assayed using the above-described GGT and iron reductase assays. Strains that appeared to have significantly altered Ggt1 activity levels were rescreened, and selected mutants (Ggt1 overexpressors or underexpressors) analyzed by Southern blotting for confirmation of transformation (data not shown), and individual colonies were purified.

Virulence assay. The mammalian cell line used in this study was RAW 264.7 (ATCC TIB-71), a murine macrophage-like cell line acquired from the American Type Culture Collection. RAW 264.7 cells were grown in RPMI medium (Cellgro, Herndon, Va.) supplemented with 10% heat-inactivated fetal calf serum (Invitrogen, Carlsbad, Calif.). H. capsulatum virulence for RAW 264.7 cells was determined using a modified protocol for determination of macrophage killing as described elsewhere (Rappleye et al., 2004). Briefly, monolayers of RAW 264.7 cells were plated at a density of 5×10⁴ cells per well in 96-well plates and allowed to adhere overnight. Next, RAW 264.7 cells were infected with growth phase-normalized H. capsulatum yeast cultures at an MOI of 5:1 (yeasts:macrophages) in 96-well plates. Culture normalization was performed on the basis of the reduced in vitro growth rates of the GGT1 overexpressing and RNAi strains, as determined previously. Cultures were started earlier based on the degree of growth rate reduction, adjusting to achieve the same culture turbidities and cell densities at the time of initiation of infection. The plates were placed at 37° C., and infection was allowed to progress for 4 h. After 4 h, uninternalized yeast cells were washed away with serum-free RPMI medium, and complete RPMI medium containing 10% fetal calf serum was added to each well. The plates were then incubated for 5 days at 37° C. in 5% CO₂-95% air. On day 5, culture medium was removed, and the remaining macrophages were lysed with a macrophage lysis solution (10 mM Tris, 1 mM EDTA, 0.05% SDS, supplemented with Protease Inhibitor Cocktail), which liberates macrophage DNA but not yeast DNA. The remaining yeast cells were removed by centrifugation. PicoGreen double-stranded DNA quantification reagent (Molecular Probes) was used to measure the amount of released macrophage DNA in each well. Data shown were collected from five independent assays.

GGT assay of macrophage cultures. RAW 264.7 macrophages were left uninfected, inoculated with heat-killed (65° C., 10 min) H. capsulatum yeasts, or infected with live H. capsulatum yeasts by a scaled-up modification of the virulence assay described above. Briefly, 2.55×10⁵ RAW 264.7 cells were seeded per cm² in 75 cm² tissue culture flasks and allowed to adhere overnight. The monolayers were then left untreated or inoculated with live or heat-killed H. capsulatum yeast cultures at an MOI of 5:1 (yeasts:macrophages). The flasks were placed at 37° C., and infection was allowed to progress for 4 h. After 4 h, uninternalized yeast cells were washed away with serum-free RPMI medium, and complete RPMI medium containing 10% fetal calf serum was added to each well. The flasks were then incubated for 5 days at 37° C. in 5% CO₂-95% air. On day 5, culture medium was removed, and the remaining macrophages were scraped off the surface, resuspended in RPMI medium and centrifuged (1200×g, 10 min). Cell pellets were lysed with the macrophage lysis solution described above and centrifuged again (1200×g, 10 min). GGT activity was determined in the resulting RAW 264.7 cell lysates according to the protocol provided above.

Statistics. For statistical analysis, Student t-test was used (SigmaPlot 2004 ver. 4.0, Systat Software Inc., San Jose, Calif.). P values less than 0.05 were considered significant.

Initial purification and identification of GSH-FeR. GSH-FeR was partly purified from concentrated culture supernatants by a combination of chromatography techniques including ion exchange chromatography, size exclusion chromatography, and chromatofocusing. Iron reductase-positive fractions were pooled, concentrated and directly subjected to trypsin digestion followed by mass spectrometry-assisted internal amino acid sequencing. The initial purification strategy used yielded a sample containing 1.2 μg protein and was a mixture of three proteins, one of which was subsequently identified as GSH-FeR. The analysis of the trypsin-generated peptide masses by tandem mass spectrometry followed by database searches revealed two major peptides detected, LGLGDTITR and IITGTVQSVINLLDR, matching the protein sequence of γ-glutamyltransferase (GGT1; E.C. 2.3.2.2). A nucleotide sequence of a putative GGT1 gene was found in the HISTO_ZY.Contig004 in the genome of H. capsulatum G217B strain (Washington University; http://www.genome.wustl.edu/tools/blast/). In addition, genomic GGT1 sequences were identified in the contig5.9 in the genome of H. capsulatum G186AR strain (Washington University) as well as in the locus HCAG_{—}03238.1 of the supercontig 3 in the genome of H. capsulatum WU24 strain (Birren et al., The Broad Institute Genome Sequencing Platform, MIT; GenBank Accession No. AAJI00000000). In order to define a start codon of the H. capsulatum GGT1, these putative genomic sequences were used to search on-line available nucleotide and protein databases of other fungal species. BLAST searches showed 61% identity and 76% similarity to Coccidioides immitis GGT as well as to genes located in genomes of various aspergilli. Intriguingly, GGT found in the genome of Aspergillus terreus was considerably longer and its putative start codon was located much further upstream in comparison to other GGT genes. Since homologies to Coccidioides and Aspergillus GGTs were based only on gene predictions and GGT homologous proteins have not been functionally confirmed or characterized in these fungi, these were used to provide frameworks for delineating the H. capsulatum sequence. Two putative GGT1 ORF sequences were identified, (ORF1 and ORF2 shown in FIG. 1A) for use in expression cloning. Both ORF sequences contained putative translation start codons ATG. One potential GGT1 ORF, corresponding to the Coccidioides homolog, consisted of 1518 nucleotides, whereas the second sequence was an upstream-extended variant harboring an extra 240 nucleotides (1758 by after an intron splicing event of the 2421-bp genomic DNA fragment), corresponding to the A. terreus homolog. Using these two sequences, two pairs of oligonucleotides were designed (GGT1.Ascl.ORF1.F and GGT1.Sbfl.ORF1.R for ORF1, and GGT1.Ascl.ORF2.F and GGT1.Sbfl.ORF1.R for ORF2, respectively) (Table 1) and were used for PCR amplification and subsequent cloning into the pLBZ1 plasmid as described below. Both PCR-amplified putative GGT1 ORFs were cloned into respective overexpression vectors, which subsequently were electroporated into a uracil-auxotrophic H. capsulatum G217Bura5-23 strain (Woods et al., 1998). The following screening of recovered transformants revealed a significant increase in Ggt1 activity, but only in the fungi episomally expressing the extended 1758-bp cDNA fragment (ORF2), indicating, thereby, the proper identification of the H. capsulatum GGT1 gene. Subsequent DNA sequencing and bioinformatics-based gene structure prediction analyses showed that the GGT1 gene of H. capsulatum G217B is organized into 6 exons of diverse length (FIG. 1A). This sequence generates the predicted 1758-bp transcript and exactly the same size could be observed after GGT1 amplification from cDNA (FIG. 1B). In turn, this transcript encodes a predicted 586-aa preproprotein of 63.1 kDa, which contains a predicted 28-amino acid secretion signal sequence and 12 putative N-glycosylation sites in its structure.

TABLE 1
Oligonucleotides used in this work

| | | |
|---|---|---|
| | | Restriction |
| Oligonucleotide | Sequence^{a} | site |

| | | | |
|---|---|---|---|
| ORF | GGT1.AscI.ORF1.F | NNNGGCGCGCCATGTATCATAGCGGTA | AscI |
| | | TCTCTGGTGG | |
| | GGT1.SbfI.ORF1.R | NNNNNNCCTGCAGGTTATTAAACAGCT | SbfI |
| | | CCTCCCGAATCCA | |
| | GGT1.AscI.ORF2.F | NNNGGCGCGCCATGTTCAACCATTCCC | AscI |
| | | TTGTC | |
| RNAi | GGT1.RNAi.KpnI.AscI.F1 | NNNGGTACCGGCGCGCCACGACATTC | KpnI/AscI |
| | | TGGGGGAGATTG | |
| | GGT1.RNAi.HindIII.R1 | NNNAAGCTTCTCTGACACATTCGGGCT | HindIII |
| | | AAGC | |
| | GGT1.RNAi.NotI.SbfI.F2 | NNNNNNGCGGCCGCCCTGCAGGACGA | NotI/SbfI |
| | | CATTCTGGGGGAGATTG | |
| | GGT1.RNAi.XbaI.R2 | NNNTCTAGACTCTGACACATTCGGGCT | XbaI |
| | | AAGC | |
| | t-1 | NNNGGATCCATCGTCCATTCCGACAGC | BamHI |
| | | ATCG | |
| | t-2 | NNNGGATCCTAGTGGCTCCAAGTAGCG | BamHI |
| | | AAGC | |

^{a}Underlined sequences show the introduced restriction enzyme site in the corresponding primer.

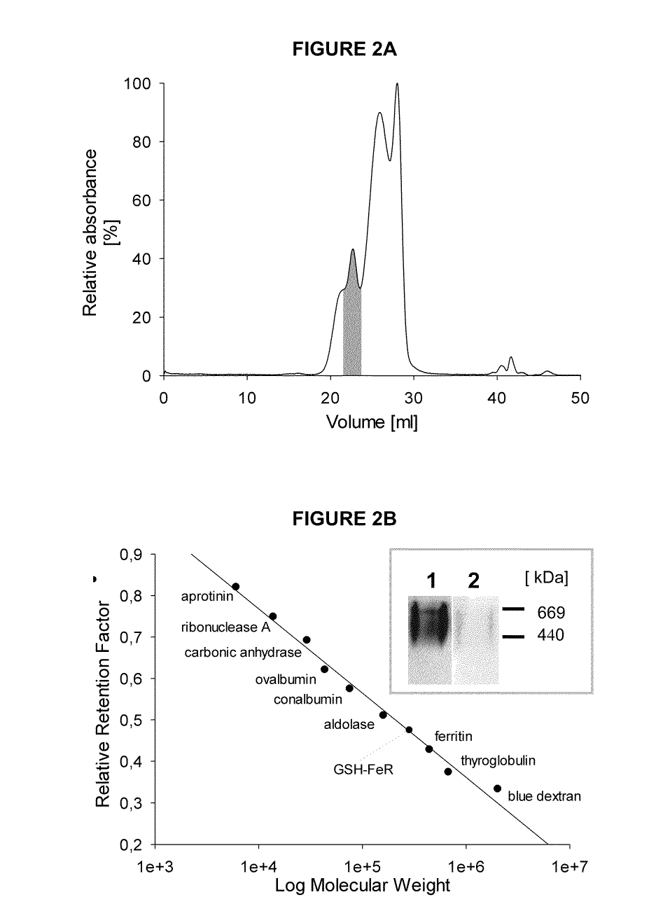

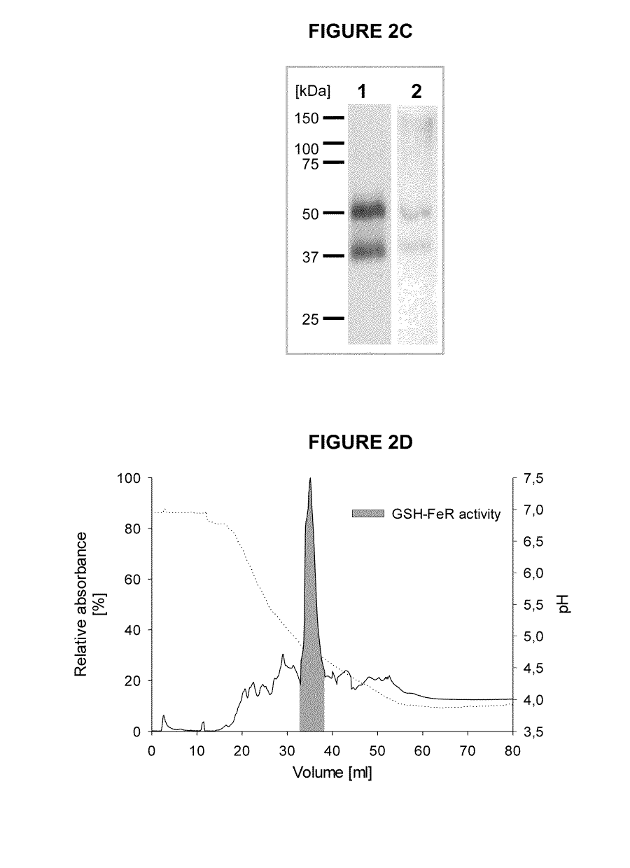

Purification and biochemical characterization of Ggt1. To purify and assess biochemical properties of Ggt1, the enzyme was overexpressed under the control of a constitutive H. capsulatum-native promoter. Purification of heterologously expressed Ggt1 was done using the same purification strategy as described above, which yielded considerably more of the pure protein (FIG. 2A). The recovery corresponded to 5.3% of the initial enzymatic activity and its specific activity increased 1304-fold (Table 2).

FIG. 2 is a chromatographic analysis of Ggt1 purified from H. capsulatum G217B culture supernatants. Proteins were detected at 280 nm. Ggt1 activity shown as a forward diagonally shaded gray area. FIG. 2(A): Purification of Ggt1 in gel filtration chromatography. Proteins were separated in PBS (pH 7.2) on two Superdex™ 200 10/300 GL columns (Amersham) set in a row. FIG. 2(B): Size exclusion column calibration with a set of protein standards: blue dextran (˜2 MDa), thyroglobulin (669 kDa), ferritin (440 kDa), aldolase (158 kDa), conalbumin (75 kDa), ovalbumin (43 kDa), carbonic anhydrase (29 kDa), ribonuclease A (13.7 kDa), and aprotinin (6 kDa). Position of Ggt1 indicated by a dotted line. Inset: Electrophoretic analysis of native Ggt1 in 10% polyacrylamide gel subsequently stained with silver (1) and for the presence of glycoproteins (2). FIG. 2(C): SDS-PAGE analysis of purified Ggt1 in 10% denaturing discontinuous SDS-polyacrylamide gel subsequently stained with silver (1) and for the presence of glycoproteins (2). FIG. 2(D): Chromatofocusing of Ggt1 on MonoP 5/200 GL. A solid line represents an elution profile of proteins separated in a pH gradient from 7 to 4 (dotted line).

On a native PAGE gel, the purified Ggt1 occurred as a very large glycoprotein complex (FIG. 2B); however subsequent high-performance chromatographic separation on a set of two Superdex 200 columns allowed a precise size of Ggt1 to be determined at 320±10% kDa. On a SDS-PAGE gel after gel filtration, the Ggt1 preparation appeared pure with two apparent equally glycosylated bands at about 50 and 37 kDa, which is a common feature of all known GGT-type enzymes (FIG. 2C). Chromatofocusing of the purified enzyme determined its isoelectrofocusing point (pI) at 4.8 (FIG. 2D).

TABLE 2
Summary of H. capsulatum Ggt1 purification

| | | | | GGT | GGT | | |
|---|---|---|---|---|---|---|---|
| | | Total | GGT | total | specific | | |
| | Protein | protein | activity | activity | activity | Yield | Fold |
| Step^{a} | [mg/ml] | [mg] | [U/ml]^{b} | [U]^{b} | [U/mg]^{b} | [%] | purified |
| Raw supernatant | 0.64 | 11508.18 | 6.1 | 109920 | 9.6 | 100 | 1 |
| Filter-concentration | 3.05 | 64.02 | 885.5 | 39846 | 622 | 36.3 | 65 |
| DEAE | 0.73 | 4.38 | 706.7 | 21201 | 4840 | 19.3 | 504 |
| Sephacryl | 0.05 | 2.76 | 378.0 | 20415 | 7397 | 18.6 | 771 |
| MonoQ/Superdex | 0.0111 | 0.4656 | 239.0 | 5829 | 12519 | 5.3 | 1304 |

^{a}Purification from 18 I batch culture.
^{b}1 U of GGT activity generates 1 nmol pNA per min

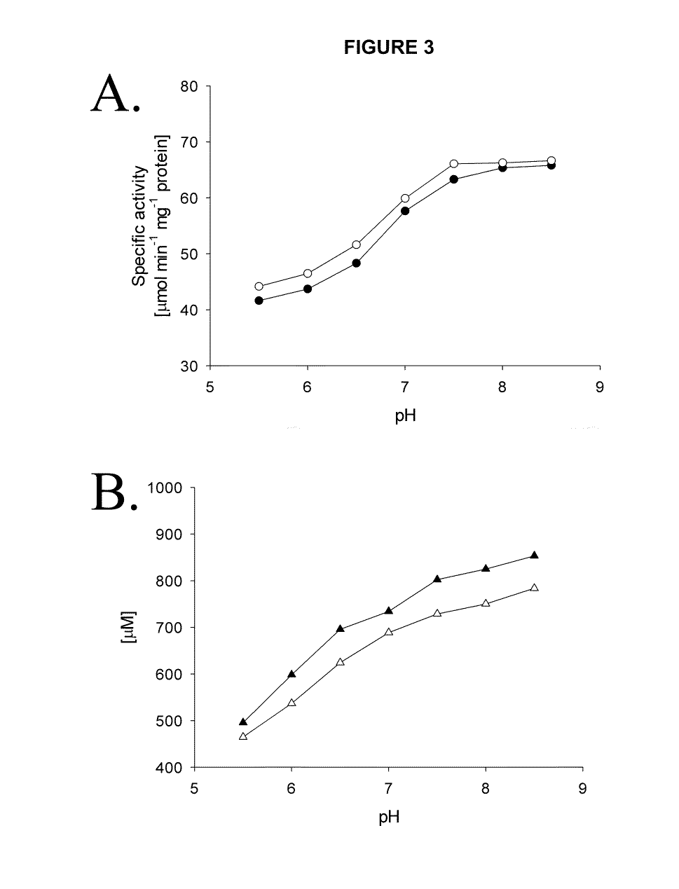

The existence of considerable discrepancies in catalytic properties between GGTs of different origins has been well documented showing certain preferences towards transpeptidation, hydrolysis or both reaction types (Ikeda et al., 1995, J. Biol. Chem. 270: 22223-22228; Hiratake, 2005, Chem. Rec. 5: 209-228; Boanca et al., 2006, J. Biol. Chem. 282: 534-541). To evaluate enzymatic properties of Ggt1 isolated from H. capsulatum cultures, a non-physiological substrate analogue γ-glutamyl-p-nitroanilide (GpNA) was used as an artificial donor of glutamyl residues. GpNA is the substrate employed in most GGT studies and its decomposition in the presence of Ggt1 can be monitored spectrophotometrically at 412 nm. 20 mM glycylglycine was also applied, which acted as an acceptor of glutamyl groups in the reaction of transpeptidation. Measurements to determine kinetic constants, Kₘ and Vₘₐₓ, were carried out using purified Ggt1 incubated with increasing concentrations of GpNA and, in the case of glutamyl transfer reaction, with glycylglycine added. Saturation kinetics was observed and the data were then processed using non-linear regression analysis. There was significant pH dependence for kinetics constants, Vₘ and Kₘ, but differences in hydrolysis and transpeptidation activities of Ggt1 were rather marginal (FIG. 3).

FIG. 3 illustrates the pH-dependent kinetics of the H. capsulatum Ggt1-catalayzed transfer and hydrolysis reactions of the artificial substrate GpNA. The maximal enzymatic activity at a saturating substrate concentration was determined in four repetitions and mean values were plotted as a function of pH. H. capsulatum Ggt1 was mostly active at neutral and slightly alkaline pH. FIG. 3(A): At the indicated pH values, Vₘ values for both glutamyl transfer from () and hydrolysis (◯) of GpNA were determined. FIG. 3(B): At the indicated pH values, Kₘ values for both glutamyl transfer from (▴) and hydrolysis (Δ) of GpNA were calculated.

The highest Ggt1 activity in the GpNA hydrolysis was observed at neutral and slightly alkaline pH and the apparent average Vₘ was 66.34±0.77 μmol min⁻¹ (mg GGT)⁻¹, whereas the apparent optimal Kₘ for GpNA was about 0.75±0.02 mM. Glutamyl transfer reaction in the presence of 20 mM glycylglycine was slightly slower with the calculated apparent Vₘ of 64.83±0.77 μmol min⁻¹ (mg Ggt1)⁻¹ and the apparent optimal Kₘ for GpNA of about 0.84±0.02 mM. Mass spectrometric analysis confirmed that the reaction of transpeptidation occurred, as a molecular peak corresponding to the molecular mass of glutamylglycylglycine was detected.

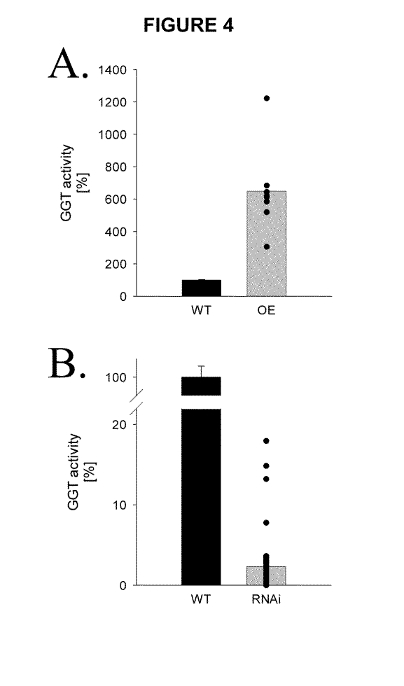

Ggt1 is involved in extracellular iron reduction in H. capsulatum. To assess the role of Ggt1 in extracellular iron reduction, a panel of H. capsulatum G217B mutants with altered GGT1 expression/activity levels was created. A strain bearing an empty pLBZ1 vector was used as a control. There was no difference in Ggt1 activity observed between the wild type G217B strain and the empty vector control transformant. Upregulation of the Ggt1 activity was achieved by expressing the GGT1 gene under the constitutive H. capsulatum-native H2B promoter (Bohse and Woods, 2007, Infect. Immun. 75: 2811-2817), which provided a considerable, about six-fold increase in the enzyme activity when measured in high-molecular weight supernatant fraction (FIG. 4A).

FIG. 4 illustrates how the Ggt1 enzymatic activity in high-molecular weight supernatant fractions of mutants with altered GGT1-expression levels. Ggt1 activity levels were normalized per protein content. FIG. 4(A): Upregulation of Ggt1 activity heterologously expressed under the constitutive native H2B promoter. The black bar represents the level of Ggt1 activity in control, the gray bar denotes the average fold change in enzymatic activity, whereas black dots represent individual transformants. FIG. 4(B): Residual Ggt1 activity in RNAi-silenced mutants. The black bar represents the mean Ggt1 activity in control, whereas the gray bar refers to the average residual enzymatic activity in RNAi-silenced mutants, whereas each black dot represents individual transformants.

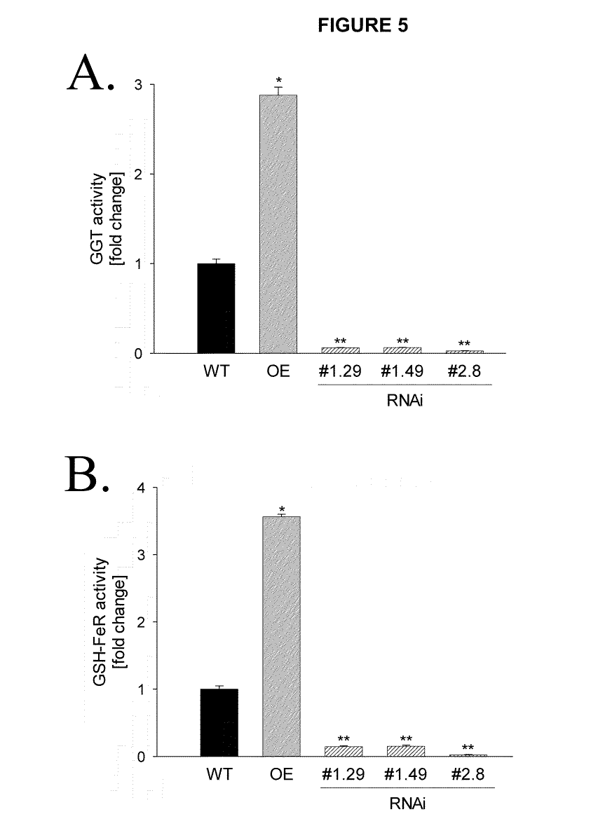

To down-regulate GGT1 expression, RNA interference-mediated silencing was successfully applied. Subsequent screening of the generated transformants showed an efficient over 40-fold reduction in Ggt1 activity in these supernatant fractions (FIG. 4B). One of the GGT1-overexpressing strains (OE) and three of the GGT1 RNAi-silenced mutants (#1.29, #1.49, and #2.8) were chosen and their ability to reduce ferric iron in cultures was then assayed. Comparing to control, cultures of the Ggt1-overproducing strain possessed nearly three-fold higher GGT activity, whereas supernatants from the selected RNAi mutants #1.29, #1.49, and #2.8 had only 6.3, 6.1, and 3.6% of GGT activity in the corresponding control, respectively (FIG. 5A). Subsequent chromatographic analyses of supernatant proteins obtained from those mutants' cultures demonstrated changes in intensities of the Ggt1-corresponding peaks, which correlated with the observed levels of the enzymatic activity (data not shown).

GSH-dependent iron reductase activity was also assessed in these strains. FIG. 5 shows the modulation of Ggt1 activity shows concordant alterations in extracellular iron reduction in H. capsulatum G217B culture supernatants. Changes in Ggt1 activity (FIG. 5A) and GSH-FeR activity (FIG. 5B) were determined in culture supernatants and normalized for protein content. Data shown are from a representative of four independent experiments carried out in triplicate. * and ** indicate significant differences in enzymatic activities (p<0.05) compared to WT.

In general, the altered patterns in GGT1 expression/activity corresponded to almost the same changes in GSH-FeR activity (FIG. 5B). RNAi-mediated downregulation of Ggt1 activity resulted in a decreased GSH-dependent extracellular iron reduction, which in the three selected RNAi transformants amounted to only 14.6, 15.0, and 3.0% of the activity observed in wild type. Upregulation of Ggt1 activity had the opposite effect and yielded about a 3.6-fold increase in GSH-FeR activity in culture supernatants. These results establish a link between Ggt1 and extracellular ferric iron reduction by H. capsulatum.

Insights into a mechanism of Ggt1-assisted extracellular iron reduction in H. capsulatum. To confirm that Ggt1 activity was responsible for these findings, a set of classical GGT inhibitors, such as serine/borate complex (SBC), 6-diazo-5-oxo-L-norleucine (DON), and acivicin, was tested (Table 3). In addition, the effects of non-reducible iron analogues, such aluminum and gallium, which are recognized as potent GSH-FeR inhibitors, on both glutamyltransferase and ferric reductase activities, were examined. Prior to each enzymatic reaction, the enzyme preparation was pretreated with individual inhibitors (used at 0.1 mM concentration) for 15 min, after which inhibitors were removed by diafiltration and proper substrates were added. Control reactions remained untreated. SBC, DON, and acivicin completely abolished both GpNA conversion and ferric iron reduction. Aluminum and gallium also exerted a strong inhibitory effect upon GSH-FeR activity; however no inhibitory action of these two metals was detected with reference to GGT activity. Based on these observations, it was concluded that extracellular ferric iron reduction in H. capsulatum is a two-step process with an initial step catalyzed by Ggt1 followed by the ferric iron reduction one.

TABLE 3
Effect of inhibitors on glutamyltransferase and iron reduction
activities of Ggt1 and cysteinylglycine

| | Target reaction catalyzed by | | | |
|---|---|---|---|---|
| | γ-Glutamyltransferase | | Cysteinylglycine | |
| | (Ggt1) | | Glu | |
| Inhibitor^{a} | Glu transfer | Fe³⁺ reduction | transfer | Fe³⁺ reduction |
| Acivicin, | Inhibition | Inhibition | No effect | No effect |
| DON^{b}, SBC^{b} | | | | |
| Aluminum, | No effect | Inhibition | No effect | Inhibition |
| gallium | | | | |

^{a}Inhibitors were used at 0.1 mM concentration.
^{b}DON, 6-diazo-5-oxo-L-norleucine; SBC, serine/borate complex.

As the secreted ferric reductase of H. capsulatum exerts its activity only when GSH is present, it is possible that the Ggt1-catalyzed process of extracellular iron reduction somehow involves utilization of GSH. In fact, GSH as a tripeptide consisting of γ-glutamyl, cysteinyl and glycinyl residues is an excellent physiological substrate, which can be efficiently metabolized by eukaryotic GGT-type enzymes (Mehdi et al., 2001, Biochem. J. 359: 631-637; Csala et al., 2003, BioFactors 17: 27-35). To verify this assumption, the composition of products generated from GSH during the course of the H. capsulatum Ggt1-catalyzed reaction was analyzed using chromatographic and spectrometric approaches. Mass spectrometry analysis revealed a significant decrease in GSH content accompanied by an additional molecular peak corresponding to the molecular mass of cysteinylglycine (data not shown). Interestingly, a molecular peak representing a glutamylglycylglycine tripeptide could be scarcely observed in transpeptidation-type reaction mixtures amended with glycylglycine. A lack of abundant amounts of glutamylglycylglycine does not mean this tripeptide was not generated in the reaction course, but rather indicates its relatively tentative nature in the presence of Ggt1 and also results from a low efficiency of transpeptidation. With all probability, this short tripeptide generated in small amounts underwent a subsequent GGT-catalyzed conversion into glycylglycine and simultaneous glutamyl residue transfer onto a water molecule. In fact, two distinct molecular peaks corresponding to these two chemicals could be seen on mass spectrometry chromatograms (data not shown). This finding is in good agreement with the above-described observations confirming that the H. capsulatum Ggt1 preferentially catalyzes hydrolysis-type reactions. Overall, GSH could be converted into cysteinylglycine in the presence of H. capsulatum Ggt1.

GSH is an important and abundant antioxidant, which can reduce ferric iron. The reduction process involves a highly reactive thiol group of cysteine. On the other hand, cysteinylglycine, the product of Ggt1-catalyzed GSH conversion, also possesses this thiol group and should possess similar reductive properties. To verify this hypothesis, the ability of both substances to reduce iron was determined. GSH and its GSH-derived dipeptide were used at concentrations up to 10 mM.

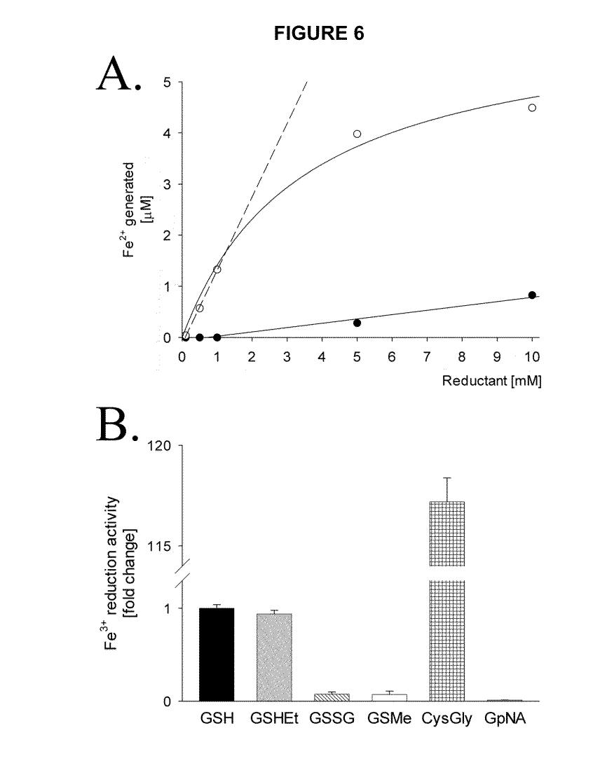

FIG. 6 shows the ferric iron reducing activities of GSH () and CysGly (◯). FIG. 6(A): Both substances were examined at concentrations up to 10 mM and amounts of generated ferrous iron were determined with ferrozine. The mean values shown are from a representative of three independent experiments carried out in triplicate. Linear regression analysis represented here demonstrated a linear correlation between iron reduction and CysGly within a concentration range of the latter up to 1.5 mM (r² coefficient of 0.99871). FIG. 6(B): Effects of chemical modification of GSH upon iron reduction. The substances GSH or GSH ethyl ester (GSHEt) possessed a free sulfhydryl (SH) group within a cysteine residue, whereas oxidized GSH (GSSG) and S-methyl-GSH (GSMe) had this group modified as a result of oxidation or alkylation, respectively. Cysteinylglycine (CysGly), which has a free SH group, was included as an extra positive control, whereas GpNA was used as a negative one. All the tested substances were used at 1 mM concentration. The mean values shown are from a representative of three independent experiments carried out in triplicate.

As shown in FIG. 6A, cysteinylglycine at as low a concentration as 0.5-1 mM (which was normally utilized throughout the entire study) generated measurable amounts of ferrous iron, whereas only slight iron reducing activity was observed with 1 mM GSH. In fact, the ability of 1 mM cysteinylglycine to reduce iron was over 117-fold higher when compared to the activity of 1 mM GSH (FIG. 6B). The differences in iron reduction activity between GSH and cysteinylglycine could be due to distinct three-dimensional structures of both compounds, which directly determine variable access to the thiol groups in those chemicals. In GSH, the thiol group is located in the middle of the molecule and is surrounded by two amino acid residues. This situation is different in cysteinylglycine, where the thiol group of cysteine is surrounded by only one glycine residue and, thereby, remains more sterically available to potential oxidized substrates.

To test whether the thiol group within the cysteinyl residue is involved in iron reduction, various derivatives of GSH with different modifications of either thiol or other functional chemical groups in their structures were examined (FIG. 6B). All the chemicals were tested at 1 mM concentration. Iron reduction activity could be easily detected when compounds bearing free SH, such as GSH or its ethyl ester (GSHEt), were studied. On the other hand, no ferrous iron was generated when oxidized derivatives of GSH, such as oxidized glutathione (GSSG) or S-methylglutathione (GSMe), were tested. These results indicated the thiol group was indispensable for iron reduction to occur. Interestingly, iron reduction activity of the cysteinylglycine dipeptide was inhibited in the presence of trivalent aluminum and gallium ions (Table 3). This inhibitory action could be overcome by raising ferric iron concentration (Liesener et al., 2004, In: Proceedings of the 104th American Society for Microbiology General Meeting, New Orleans, La., May 23-27, 2004. Abstract F-066), which indicated a competitive nature of these two metal inhibitors. In fact, iron and its non-reducible trivalent analogues are able to form relatively stable complexes with thiol-containing amino acid and short oligopeptides (Farkas and Sovago, 2002, In: Amino acids, Peptides and Proteins, vol. 32, Barrett and Davies, eds, Cambridge: Royal Society of Chemistry Publishing, pp. 295-364).

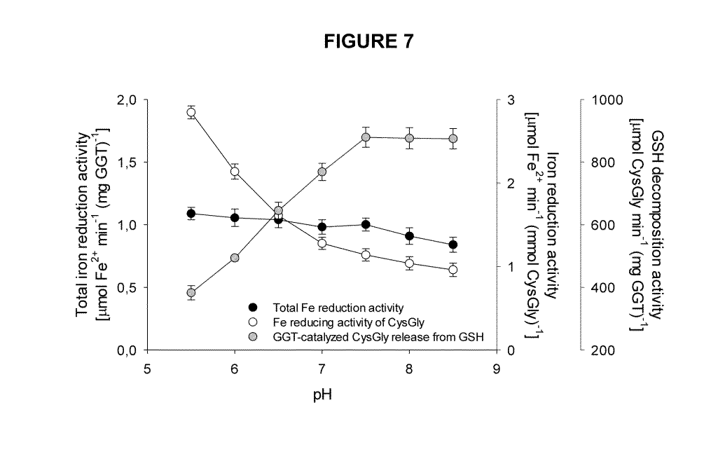

Kinetics of the Ggt1-assisted extracellular iron reduction in H. capsulatum. To evaluate the efficacy of Ggt1-assisted iron reduction as a function of pH and to determine the efficiency of Ggt1-catalyzed decomposition of GSH (which is equivalent to release ratios of cysteinylglycine from GSH), the following approach was applied. Firstly, the total iron reduction capability of this reaction system was measured (with both Ggt1 and GSH added). Then, the extent of iron reduction by cysteinylglycine alone was determined. Both determinations were carried out in a broad pH range from 5.5 to 8.5, which enabled detailed calculations of kinetic parameters of the Ggt1-catalyzed reaction of GSH hydrolysis. Total iron reduction activity was almost constant in the range of pH values examined and a diminutive, but statistically insignificant trend toward decreasing this activity at higher pH could be observed (FIG. 7).

Shown in FIG. 7 is the process of Ggt1-catalyzed extracellular iron reduction is not dependent on pH due to complementary contribution of Ggt1 and cysteinylglycine activities. Total iron reduction activity is generally constant in a broad pH range and tends to decrease slightly under alkaline conditions. This phenomenon results from a deft combination of kinetics parameters of both reactions involved in this process. Cysteinylgycine is mostly active at lower pHs, whereas its low activity at neutral and slightly alkaline conditions is compensated by higher activity of Ggt1 and is accompanied by more intensive release of this dipeptide from GSH. At lower pH, Ggt1 is less active and generates less molecules of cysteinylglycine, which in turn are more active in acidic environments.

The maximal iron reduction of 1.09±0.05 μmol of Fe²⁺ min⁻¹ (mg Ggt1)⁻¹ was determined in the test at pH 5.5, whereas the lowest amounts of reduced ferrous iron (0.84±0.06 μmol of Fe²⁺ min⁻¹ (mg Ggt1)⁻¹) were measured at pH 8.5. This even level of iron reduction activity at different pHs resulted from differences in activities of both Ggt1 and cysteinylglycine. The latter was mostly active in acidic environments (2.85±0.08 μmol of Fe²⁺ min⁻¹ (mmol CysGly)⁻¹ at pH 5.5) and considerably less efficient as an iron-reducing factor under neutral and alkaline conditions (0.96±0.08 μmol of Fe²⁺ min⁻¹ (mmol CysGly)⁻¹). The opposite situation was observed with reference to the Ggt1 activity characteristics computed with GpNA (as reported in FIG. 3). The same Ggt1 activity pattern could be observed in the case of GSH hydrolysis. This reaction was optimal at pH above 7.5 and released up to 0.88±0.03 mmol of cysteinylglycine min⁻¹ (mg Ggt1)⁻¹ (which is equivalent to decomposition of the same amount of GSH). Based on these findings, it was concluded that the reaction catalyzed by cysteinylglycine was rate limiting at higher pH and was compensated by an increase in Ggt1 activity, and vice versa at lower pHs. Overall, the data indicated that the efficiency of extracellular iron reduction in H. capsulatum resulted from a combination of both reactions complementing one another in this process.

Ggt1 and H. capsulatum in vitro growth and virulence. To determine whether Ggt1 contributes to the H. capsulatum virulence, the panel of created GGT1-mutant strains for growth at 37° C. in HMM, a rich defined medium, was first assayed. Equal numbers of yeast cells taken from late-log-phase cultures (as determined for wild type) were grown for 4 days and culture turbidity was monitored in 12 h intervals.

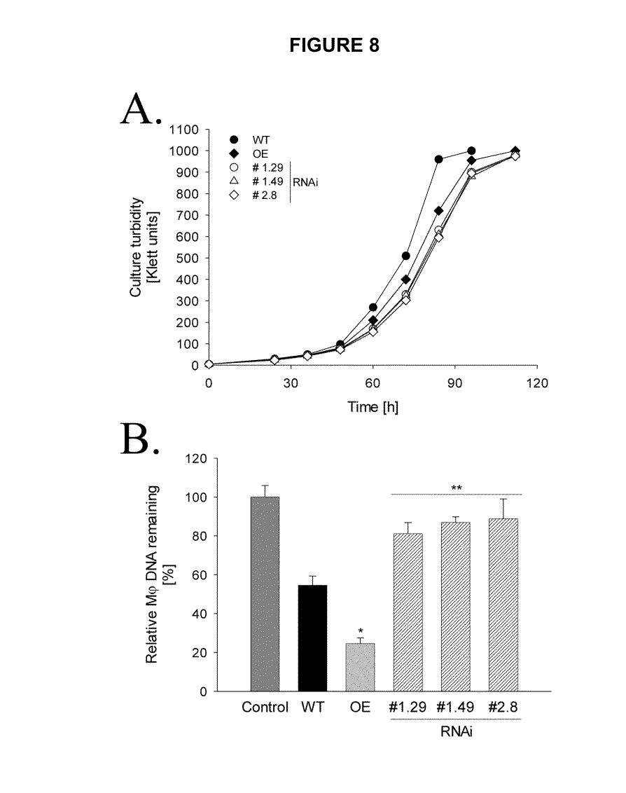

Shown in FIG. 8 are changes in GGT1 expression alter H. capsulatum growth and virulence. (A) GGT1 overexpression or downregulation in RNAi mutants both impair fungal growth in vitro in rich defined HMM. Growth was assessed based on culture turbidity measured with a Klett-Summerson photometric colorimeter. Data are shown for a representative experiment from five independent experiments with similar results. (B) Variations of GGT1 expression are correlated with the virulence of H. capsulatum in RAW 264.7 macrophages. Macrophage killing is determined as reductions in macrophage DNA concentration remaining after incubation of macrophages with H. capsulatum yeasts. Macrophage DNA remaining at 5 days after infection was compared to uninfected wells (Control). Data shown were collected from five independent assays. * and ** indicate significant differences (p<0.05) compared to WT.

Alterations in H. capsulatum Ggt1 activity caused substantial defects in the in vitro growth of both GGT1 overexpressing and RNAi silenced mutants (FIG. 8A). Although all cultures achieved the same final turbidity, log-phase growth rates of the GGT1 overexpressing strain were reduced approximately by 22-25% relative to the wild-type control, whereas the RNAi mutants showed about 34-37% reduction in log-phase growth. Because these H. capsulatum strains grew in vitro at different rates, growth phase-normalized yeast cultures in infection assays were utilized. The relative virulence was determined by infecting murine macrophage-like RAW 264.7 cells with the H. capsulatum strains and subsequently measuring host cell death. Alterations in Ggt1 activity correlated with H. capsulatum virulence in this infection model. Compared to uninfected macrophage controls, both wild type and empty vector control strains caused substantial damage to macrophage monolayers (shown as WT in FIG. 8B). Relative to these WT controls, the GGT1 overexpressing mutant appeared more virulent despite its reduced growth rate alone in HMM, whereas RNAi downregulation of GGT1 yielded significant deficits in virulence (FIG. 8B). Similar results were obtained in five independent experiments.

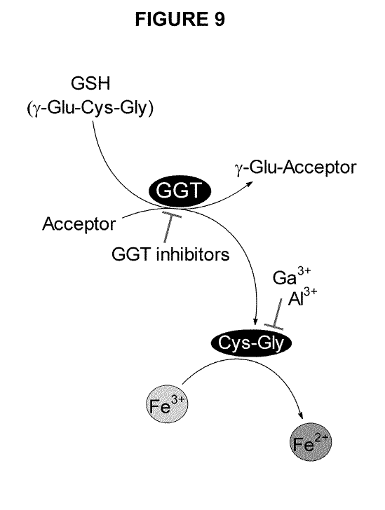

Not wanting to be bound by the following theory, a proposed model of Ggt1-assisted extracellular iron reduction in H. capsulatum involves two basic reactions, and is illustrated in FIG. 9. The first step is strictly enzymatical and requires the presence of both GSH and Ggt1. GSH is the major thiol found in animal cells at millimolar levels and is one of the most abundant glutamyl peptides existing in nature, which is a potential source of nitrogen and sulfur. This ubiquitous substance is also present in macrophages at concentration ranging up to 10 mM, which potentially makes GSH available to H. capsulatum during infection. In fact, lung tissues are the richest in GSH in the human body, where this thiol is present at levels about 100 times higher than those observed in plasma. In the first step, secreted Ggt1 initiates the enzymatic breakdown of extracellular GSH by cleavage of the γ-glutamyl bond and releasing cysteinylglycine. In the second step, the thiol group of the released cysteinylglycine dipeptide interacts with ferric ions and this interaction results in generation of ferrous iron. Both reactions contribute to the efficiency of iron reduction and each step may become rate limiting under certain circumstances; however, the resultant total iron reduction activity of this system is generally constant in a broad pH range. This phenomenon results from a deft combination of kinetic parameters of both reactions involved in this process. Cysteinylglycine is mostly active at lower pH, whereas its low activity at neutral and slightly alkaline conditions is compensated by higher Ggt1 activity rates and is accompanied by more intensive release of this dipeptide from GSH. Conversely, Ggt1 is less active at lower pH, and thereby generates less molecules of cysteinylglycine, which in turn are more active in acidic environments and balance the iron reduction yield. This finding indicates that the process of iron reduction may occur under H. capsulatum infection-related conditions in the host macrophages. The definite preference of H. capsulatum yeast cells to acquire iron in its reduced more soluble ferrous form also supports this statement (Zarnowski et al., 2008, Curr. Microbiol. 57: 153-157).

The purified H. capsulatum Ggt1 shared some enzymatic properties with other known GGTs, but also was different from any of the enzymes that have been reported to possess iron reductase activities. Genetic manipulations of the GGT1 expression leading to either upregulation or downregulation of the corresponding glutamyltransferase activity concurrently resulted in a significant increase or in a significant decrease in iron reducing activity in H. capsulatum cultures, indicating that Ggt1 functions as a leading factor in extracellular iron reduction in this pathogenic fungus. The ferric reduction activity could also be abolished in the presence of classical GGT inhibitors, such as SBC, DON, or acivicin, which provide a link between these two activities. Additional tests involving aluminum and gallium ions demonstrated iron reduction to be a complex two-step process. The iron reduction-associated function of H. capsulatum Ggt1 is a newly identified physiological role for GGT-type proteins. As GSH-dependent iron reduction activity is common to all H. capsulatum var. capsulatum and H. capsulatum var. duboisii strains and also to three other species of dimorphic zoopathogenic fungi, including Blastomyces dermatitidis, Paracoccidioides brasiliensis, and Sporothrix schenckii (Zarnowski and Woods, 2005), the results presented herein suggest that all these species utilize secreted GGT enzymes to generate reduced iron.

The efficiency of Ggt1-catalyzed iron reduction in H. capsulatum was assessed under acidic and neutral environments, because the fungus faces these pH conditions in the course of infection in a unique phagosomal or phagolysosomal compartment within pulmonary macrophages (Newman et al., 2006, J. Immunol. 176: 1806-1813). The enzymatic ferric iron reduction activity in H. capsulatum G217B cultures was similar over a broad pH range (Zarnowski and Woods, 2005). In this work, the process of Ggt1-assisted ferric iron reduction was examined in the presence of GSH, which provided results consistent with the previous observations.

Alterations in H. capsulatum Ggt1 activity caused substantial defects in the in vitro growth rates of both GGT1 overexpressing and RNAi silenced mutant strains in the defined rich medium HMM. The attempt to complement the observed in vitro growth defect by providing extra amounts of ferrous or ferric or transferrin-bound iron failed to restore growth rates of the mutants (data not shown). The observed in vitro growth differences are due to a GGT1 over- or underexpression effect unrelated to iron, which is not surprising given the potential roles of the enzyme in redox reactions as well as nitrogen and sulfur metabolism. This is supported by the relatively high Kₘ values reported in this study. If the Kₘ for the Ggt1-catalysed decomposition of GSH were low, this enzymatic process would be too intense and would result not only in decreased GSH amounts, but would also lead to proportionate accumulation of CysGly followed by excessive iron reduction in the Histoplasma-infected macrophages. Thus, the redox balance within this unique microenvironment would be impaired. In fact, it is likely that this phenomenon occurs in the GGT1 overexpressing mutant, thereby underlying the observed growth defect in this strain.

GGT is one of a number of virulence factors produced by H. pylori. As H. capsulatum is an intracellular pathogen, the contribution of Ggt1 to virulence was determined using the murine macrophage-like RAW 264.7 cell line as an infection model. The experiments to overexpress or underexpress GGT1 provided evidence linking Ggt1 activity levels to H. capsulatum virulence. The GGT1 overexpressing strain killed more macrophages in vitro than wild type or empty vector control strains, whereas this macrophage killing capability was significantly reduced in the GGT1 RNAi mutants. GGT activity was detected in cultures of macrophages infected with live H. capsulatum, but not uninfected macrophage cultures or cultures inoculated with heat-killed fungal cells (data not shown). The kinetic properties of this enzymatic activity were the same as determined for H. capsulatum Ggt1 (data not shown), which is consistent with production of this enzyme by the pathogenic fungus in infected RAW 264.7 macrophages.

Finally, the recent report of H. capsulatum secretory vesicles (Albuquerque et al., 2008) provides a potential mechanism for extracellular transport and delivery of concentrated enzymes, which in the case of Ggt1 would foster the generation of CysGly and consequent reduction of iron.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of biochemistry and molecular biology, are within the scope of this invention. All publications, patents, and patent applications cited herein are incorporated by reference in their entirety for all purposes.

What is claimed is:

1. An isolated polynucleotide encoding a polypeptide comprising an amino acid sequence that is at least 95% identical to the amino acid sequence of SEQ ID NO:2, wherein the polypeptide has a γ-glutamyltransferase activity.

2. The isolated polynucleotide of claim 1, which is at least 95% identical to the nucleic acid sequence of SEQ ID NO:1.

3. The isolated polynucleotide of claim 1, which encodes the amino acid sequence of SEQ ID NO:2.

4. The isolated polynucleotide of claim 1, which is isolated from Histoplasma capsulatum.

5. A polynucleotide comprising the nucleotide sequence of the antisense strand of the polynucleotide of claim 1 or a part thereof.

6. A vector comprising the polynucleotide of claim 1.

7. The vector of claim 6 further comprising a recombinant expression cassette, which comprises a promoter sequence operably linked to the polynucleotide.

8. A host cell transformed with a vector comprising the polynucleotide of claim 1.

9. An isolated polypeptide comprising an amino acid sequence that is at least 95% identical to the amino acid sequence of SEQ ID NO:2, wherein the polypeptide has a γ-glutamyltransferase activity.

10. The isolated polypeptide of claim 9, which polypeptide is extracellularly secreted.

11. The isolated polypeptide of claim 9, which is isolated from Histoplasma capsulatum.

12. An antibody immunologically specific for the polypeptide of claim 9.

13. A transgenic organism comprising a recombinant expression cassette, which comprises a promoter sequence operably linked to a polynucleotide encoding a polypeptide, wherein the polypeptide comprises an amino acid sequence that is at least 95% identical to the amino acid sequence of SEQ ID NO:2.

14. The transgenic organism according to claim 13, which exhibits increased γ-glutamyl transfer activity over the corresponding non-transgenic organism.

15. The transgenic organism according to claim 13, which is transgenic Histoplasma capsulatum.

16. A method, comprising
reacting a polypeptide comprising an amino acid sequence that is at least 95% identical to the amino acid sequence of SEQ ID NO:2 with glutathione to form cysteinylglycine; and
reacting the cysteinylglycine with ferric iron (Fe³⁺) to form ferrous iron (Fe²⁺).

17. The method of claim 16 further comprising the step of measuring the amount of formed ferrous iron, the amount of ferrous iron being proportional to γ-glutamyl transfer activity of the polypeptide.

18. The method of claim 16, wherein the method is performed extracellularly.

19. The method of claim 16 wherein the polypeptide is a γ-glutamyl transferase from Histoplasma capsulatum.

20. A kit for use in an analysis of iron reduction in a sample, the kit comprising:
a) a polypeptide comprising an amino acid sequence that is at least 95% identical to the amino acid sequence of SEQ ID NO:2; and
b) glutathione,
wherein the polypeptide and the glutathione are present in one or more containers, and
wherein the polypeptide has a γ-glutamyltransferase activity.

21. The kit of claim 20 wherein the polypeptide is a γ-glutamyl transferase from Histoplasma capsulatum.

22. A target for fungicides, comprising
a) a nucleic acid sequence having the nucleic acid sequence depicted in SEQ ID NO:1; or
b) a nucleic acid sequence which can be derived by back-translation from the amino acid sequence of SEQ ID NO:2, due to degeneracy of the genetic code; or
c) a functional equivalent of the nucleic acid sequence of SEQ ID NO:1, which is at least 95% identical to SEQ ID NO:1.

23. A method for identifying substances with antifungal activity, wherein transcription, expression, translation, or activity of the gene product of an amino acid sequence encoded by a polynucleotide according to claim 1 is influenced, and
those substances which reduce or block transcription, expression, translation or activity of the gene product are selected.


SEQUENCE LISTING (11 sequences)

SEQ ID NO: 1 - DNA, 1758 bp, Histoplasma capsulatum
atgttcaaccattcccttgtcgtcgtcgtctcggcggcatcgatgatgctgctcattgcggtttctctatactcgtcccttacgacattctgggggagattggacgtgcagctgagcagcgatgcttctgctcctggaaatggccgtcatggtcagttgggtgctgttgcgtctgagaatcgaaactgtagccagatgggggcggagatgatgaagatgggtggaaccgcggcggatgctatggttgcaactgtattctgcattggcgtgattgcaatgtatcatagcggtatctctggtggtggattcgcaattgtacgaacgccagaaggcgagtacgaggtcgtcgatttcagagaaatggctcctgcagcagcgcataaggatatgtataaaggtaacgtggaaggggcggttttagggggtttggctagcgctgtccctggtgagattcgagggctgcaatacctacatgagaaatatggtactttgccctggtcaacagttatgcaaccggccattcgtcttgcacgggaaggatggcccgtcaatgtggacctggtcaattacatggaggctgcaacagcaggaaaagaggattttctatccaaagatccttcatgggcggttgactttgcccccaatggaacgcggttgggcctgggggatactatcactcgtgcacgatattctcaaacactcgagacaatcgcccattacgggcccgatgctttttattctgggccgcttgcggattccatgatccgagccctccaggcacataatgggactatgactctagacgatttgaagaactatagcgttgtgattcgaaacgcttcacatatcaattatcggggttacactgtgaccagcacgagggccccgtccagtggtgtcgttgcattgagtatcttgaacattgtcggtcaatatgatgatttctttgtcaagggaaatgtgaacctgagcacccatcatcttgtcgaggcaatgagatttgcctatggacagcggacgcatctggctgacccatcctattcggagggaatggatgaatatgaagatcatatgctaaatagttccatggctgctattatccgaaacaaaatctctgattcgcatactctcaatgtctctgattataaccccaatggatttgaaagcctagaaactccgggaacttctcacgtcgtcgccgccgactccagaggcctcaccatcagcctcaccacaaccataaacctcctcttcggaagcaaagtcatggttcccgaaaccggcattatcatgaacaacgacatggatgacttttccatccccggcaccaacaactcattcggctacatcccctcacctgccaacttcatcgaaccgggcaagcgcatgctctcttccatatgcccggtgatcgtcacccatcccaacggcacggtctatttcatcacgggtgccgcgggtgggtcgcgtatcatcactggcaccgtgcagagtgtcatcaatctccttgacaggaatatgaccacctatgacgcgctcaaggaaccccgtgtccatgaccagcttagcccgaatgtgtcagagtttgagtattcgtttgataacagcacggtggcgtttatgatcagtaggaatcattcggtgaaatgggttcttccgggggccagcaaggtgcaagctatccggctgatggcggatggcatgtatgaagcggttggggagcctagacaggttgattcgggaggagctgttgcgtga

SEQ ID NO: 2 - protein, 585 aa, Histoplasma capsulatum
MFNHSLVVVVSAASMMLLIAVSLYSSLTTFWGRLDVQLSSDASAPGNGRHGQLGAVASENRNCSQMGAEMMKMGGTAADAMVATVFCIGVIAMYHSGISGGGFAIVRTPEGEYEVVDFREMAPAAAHKDMYKGNVEGAVLGGLASAVPGEIRGLQYLHEKYGTLPWSTVMQPAIRLAREGWPVNVDLVNYMEAATAGKEDFLSKDPSWAVDFAPNGTRLGLGDTITRARYSQTLETIAHYGPDAFYSGPLADSMIRALQAHNGTMTLDDLKNYSVVIRNASHINYRGYTVTSTRAPSSGVVALSILNIVGQYDDFFVKGNVNLSTHHLVEAMRFAYGQRTHLADPSYSEGMDEYEDHMLNSSMAAIIRNKISDSHTLNVSDYNPNGFESLETPGTSHVVAADSRGLTISLTTTINLLFGSKVMVPETGIIMNNDMDDFSIPGTNNSFGYIPSPANFIEPGKRMLSSICPVIVTHPNGTVYFITGAAGGSRIITGTVQSVINLLDRNMTTYDALKEPRVHDQLSPNVSEFEYSFDNSTVAFMISRNHSVKWVLPGASKVQAIRLMADGMYEAVGEPRQVDSGGAVA

SEQ ID NO: 3 - DNA, 37 bp, Artificial Sequence
  Primer GGT.AscI.ORF1.F
nnnggcgcgccatgtatcatagcggtatctctggtgg

SEQ ID NO: 4 - DNA, 40 bp, Artificial Sequence
  Primer GGT.SbfI.ORF1.R
nnnnnncctgcaggttattaaacagctcctcccgaatcca

SEQ ID NO: 5 - DNA, 32 bp, Artificial Sequence
  Primer GGT.AscI.ORF2.F
nnnggcgcgccatgttcaaccattcccttgtc

SEQ ID NO: 6 - DNA, 38 bp, Artificial Sequence
  Primer GGT.RNAi.KpnI.AscI.F1
nnnggtaccggcgcgccacgacattctgggggagattg

SEQ ID NO: 7 - DNA, 31 bp, Artificial Sequence
  Primer GGT.RNAi.HindIII.R1
nnnaagcttctctgacacattcgggctaagc

SEQ ID NO: 8 - DNA, 43 bp, Artificial Sequence
  Primer GGT.RNAi.NotI.SbfI.F2
nnnnnngcggccgccctgcaggacgacattctgggggagattg

SEQ ID NO: 9 - DNA, 31 bp, Artificial Sequence
  Primer GGT.RNAi.XbaI.R2
nnntctagactctgacacattcgggctaagc

SEQ ID NO: 10 - DNA, 31 bp, Artificial Sequence
  Primer t-1
nnnggatccatcgtccattccgacagcatcg

SEQ ID NO: 11 - DNA, 31 bp, Artificial Sequence
  Primer t-2
nnnggatcctagtggctccaagtagcgaagc